(19) United States
(12) Patent Application Publication
(10) Pub. No.: US 2010/0189656 A1
(43) Pub. Date: Jul. 29, 2010

(54) CONTRAST AGENTS

(76) Inventors: Lars-Goran Wistrand, Oslo (NO); Duncan George Wynn, Buckinghamshire (GB); Ian Martin Newington, Buckinghamshire (GB); Veronique Morisson-Iveson, Buckinghamshire (GB)

Correspondence Address: GE HEALTHCARE, INC., IP DEPARTMENT 101 CARNEGIE CENTER, PRINCETON, NJ, 08540-6231, US

(21) Appl. No.: 12/664,905

(22) PCT Filed: Jun. 27, 2008
(86) PCT No.: PCT/NO2008/000242

§ 371 Date: Dec. 16, 2009

(30) Foreign Application Priority Data
Jun. 29, 2007 (NO) 20073382

(51) Int. Cl. A61K 49/04; C07C 233/77; A61P 43/00

(52) U.S. Cl. 424/9.4; 564/153

(57) ABSTRACT

The present invention relates to a class of compounds and to diagnostic compositions containing such compounds where the compounds are iodine containing compounds. More specifically the iodine containing compounds are chemical compounds containing a triamine alkyl central moiety allowing for the arrangement of three iodinated phenyl groups bound thereto. The invention also relates to the use of such diagnostic compositions as contrast agents in diagnostic imaging and in particular in X-ray imaging and to contrast media containing such compounds.

### TECHNICAL FIELD OF THE INVENTION

The present invention relates to a class of compounds and to diagnostic compositions containing such compounds where the compounds are iodine containing compounds. More specifically the iodine containing compounds are chemical compounds containing a triamine alkyl central moiety allowing for the arrangement of three iodinated phenyl groups bound thereto.

The invention also relates to the use of such diagnostic compositions as contrast agents in diagnostic imaging and in particular in X-ray imaging and to contrast media containing such compounds.

### DESCRIPTION OF RELATED ART

All diagnostic imaging is based on the achievement of different signal levels from different structures within the body. Thus in X-ray imaging for example, for a given body structure to be visible in the image, the X-ray attenuation by that structure must differ from that of the surrounding tissues. The difference in signal between the body structure and its surroundings is frequently termed contrast and much effort has been devoted to means of enhancing contrast in diagnostic imaging since the greater the contrast between a body structure and its surroundings the higher the quality of the images and the greater their value to the physician performing the diagnosis. Moreover, the greater the contrast the smaller the body structures that may be visualized in the imaging procedures, i.e. increased contrast can lead to increased spatial resolution.

The diagnostic quality of images is strongly dependent on the inherent noise level in the imaging procedure, and the ratio of the contrast level to the noise level can thus be seen to represent an effective diagnostic quality factor for diagnostic images.

Achieving improvement in such a diagnostic quality factor has long been and still remains an important goal. In techniques such as X-ray, magnetic resonance imaging (MRI) and ultrasound, one approach to improving the diagnostic quality factor has been to introduce contrast enhancing materials formulated as contrast media into the body region being imaged.

Thus in X-ray early examples of contrast agents were insoluble inorganic barium salts which enhanced X-ray attenuation in the body zones into which they distributed. For the last 50 years the field of X-ray contrast agents has been dominated by soluble iodine containing compounds. Commercial available contrast media containing iodinated contrast agents are usually classified as ionic monomers such as diatrizoate (marketed e.g. under the trade mark Gastrografen™), ionic dimers such as ioxaglate (marketed e.g. under the trade mark Hexabrix™), nonionic monomers such as iohexyl (marketed e.g. under the trade mark Omnipaque™) iopamidol (marketed e.g. under the trade mark Isovue™), iomeprol (marketed e.g. under the trade mark Iomeron™) and the non-ionic dimer iodixanol (marketed under the trade mark Visipaque™).

The most widely used commercial non-ionic X-ray contrast agents such as those mentioned above are considered safe. Contrast media containing iodinated contrast agents are used in more that 20 millions of X-ray examinations annually in the USA and the number of adverse reactions is considered acceptable. However, since a contrast enhanced X-ray examination will require up to about 200 ml contrast media administered in a total dose, there is a continuous drive to provide improved contrast media.

The utility of the contrast media is governed largely by its toxicity, by its diagnostic efficacy, by adverse effects it may have on the subject to which the contrast medium is administered, and by the ease of storage and ease of administration. Since such media are conventionally used for diagnostic purposes rather than to achieve direct therapeutic effect, it is generally desirable to provide media having as little as possible effect on the various biological mechanisms of the cells or the body as this will lead to lower toxicity and lower adverse clinical effect. The toxicity and adverse biological effects of a contrast medium are contributed to by the components of the formulation medium, e.g. the solvent or carrier as well as the contrast agent itself and its components such as ions for the ionic contrast agents and also by its metabolites.

The major contributing factors to the toxicity of the contrast medium are identified as the chemotoxicity of the contrast agent, the osmolality of the contrast medium and the ionic composition or lack thereof of the contrast medium.

Desirable characteristics of an iodinated contrast agent are low toxicity of the compound itself (chemotoxicity), low viscosity of the contrast medium wherein the compound is dissolved, low osmolality of the contrast medium and a high iodine content (frequently measured in mg iodine per ml of the formulated contrast medium for administration). The iodinated contrast agent must also be completely soluble in the formulation medium, usually an aqueous medium, and remain in solution during storage.

The osmolalities of the commercial products, and in particular of the non-ionic compounds is acceptable for most media containing dimers and non-ionic monomers although there is still room for improvement. In coronary angiography for example, injection into the circulatory system of a bolus dose of contrast medium has caused severe side effects. In this procedure contrast medium rather than blood flows through the system for a short period of time, and differences in the chemical and physiochemical nature of the contrast medium and the blood that it replaces can cause undesirable adverse effects such as arrhythmias, QT prolongation and reduction in cardiac contractive force. Such effects are seen in particular with ionic contrast agents where osmotoxic effects are associated with hypertonicity of the injected contrast medium. Contrast media that are isotonic or slightly hypotonic with the body fluids are particularly desired. Low osmolar contrast media have low renal toxicity which is particularly desirable. The osmolality is a function of the number of particles per volume unit of the formulated contrast medium.

To keep the injection volume of the contrast media as low as possible it is highly desirable to formulate contrast media with high concentration of iodine/ml, and still maintain the osmolality of the media at a low level, preferably below or close to isotonicity. The development of non-ionic monomeric contrast agents and in particular non-ionic bis(triiodophenyl) dimers such as iodixanol (EP patent 108638) has provided contrast media with reduced osmotoxicity allowing contrast effective iodine concentration to be achieved with hypotonic solution, and has even allowed correction of ionic imbalance by inclusion of plasma ions while still maintaining the contrast medium Visipaque™ at the desired osmolality (WO 90/01194 and WO 91/13636).

The X-ray contrast media at commercial high iodine concentration have relative high viscosity, ranging from about 15 to about 60 mPas at ambient temperature. Generally, contrast media where the contrast enhancing agent is a dimer has higher viscosity than the corresponding contrast media where the contrast enhancing agent is the monomer corresponding to the dimer. Such high viscosities may pose problems to the administrators of the contrast medium, requiring relatively large bore needles or high applied pressure, and are particularly pronounced in pediatric radiography and in radiographic techniques which require rapid bolus administration, e.g. in angiography.

X-ray contrast agents of high molecular weight have been proposed for many years, for example ionic contrast agents as disclosed in U.S. Pat. No. 3,378,338. More recently polymers with substituted triiodinated phenyl groups grafted on the polymer are proposed in EP 354836, EP 436316 and U.S. Pat. No. 5,019,370. Further, WO 9501966, EP 782563 and U.S. Pat. No. 5,817,873 read on compounds having e.g. 3 and 4 substituted triiodinated phenyl groups arranged linearly or around a central core. In particular, WO 9501966 proposes compounds where three iodinated phenyl groups are arranged around the central core structure N—[—CH₂—CH₂—NH₂]₃, see page 11 and 15 of this document. However, none of these proposed compounds are on the market.

Hence there still exists a desire to develop contrast agents that solves one or more of the problems discussed above. Such agents should ideally have improved properties over the soluble iodine containing compounds on the market in one or more of the following properties: renal toxicity, osmolality, viscosity, solubility, injection volumes/iodine concentration and attenuation/radiation dose.

### SUMMARY OF THE INVENTION

The present invention provides compounds useful as contrast media having improved properties over the known media with regards to at least one of the following criteria osmolality (and hence the renal toxicity), viscosity, iodine concentration and solubility. The contrast media comprises iodine containing contrast enhancing compounds where iodine containing compounds are chemical compounds containing a central aliphatic moiety, allowing for the arrangement of three iodinated phenyl groups bound to thereto through linker groups containing amide functions. The iodine containing contrast enhancing compounds can be synthesized from commercially available and relatively inexpensive starting materials.

### DETAILED DESCRIPTION OF THE INVENTION

The new compounds of the invention, their use as X-ray contrast agents, their formulation and production are specified in the attached claims and in the specification hereinafter.

The contrast enhancing compounds are synthetic chemical compounds of formula (I)

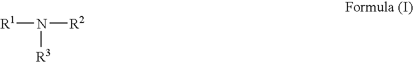

wherein R¹ and R² independently are the same or different and denote a moiety —X—NR⁴—CO—R;
each R³ denote a moiety —CO—R;
each R⁴ are the same or different and denote a hydrogen atom, hydroxyl group or a C₁-C₄ alkyl group where the alkyl group may be substituted by hydroxyl and amino groups and interrupted by an oxygen atom;
each X are the same or different and denote a straight or branched C₁-C₄ alkylene group where the alkylene group may be substituted by hydroxyl and amino groups and interrupted by an oxygen atom;
and
each R independently are the same or different and denote a triiodinated phenyl group, preferably a 2,4,6-triiodinated phenyl group further substituted by two groups R⁵ wherein each R⁵ are the same or different and denote a hydrogen atom or a non-ionic hydrophilic moiety, provided that at least one R⁵ group in the compound of formula (I) is a hydrophilic moiety;
and salts or optical active isomers thereof.

The substituents R¹ and R² above are the same or different. Preferably X denotes an unsubstituted alkylene group and most preferably a ethylene group or n-propylene group, and R¹ and R² then independent of each other denote the moiety —(CH₂)ₙ—N(R⁴)—CO—R wherein n denote an integer of 1 to 4, preferable 2 and 3. Also preferred R⁴ denotes a hydrogen atom or an unsubstituted C₁-C₄ alkyl group, preferably a hydrogen atom or a methyl group. It is also preferred that R⁴ and n are the same, and also that both of the R¹ and R² groups are the same and denote moiety of formula —(CH₂)ₙ—N(R⁴)—CO—R wherein n, R⁴ and R are as defined above.

Each of the three iodinated R groups in the compound of formula (I) can be the same or different and preferably denote a 2,4,6-triiodinated phenyl group, further substituted by two groups R⁵ in the remaining 3 and 5 positions in the phenyl moiety.

The non-ionic hydrophilic moieties may be any of the non-ionizing groups conventionally used to enhance water solubility. Hence, the R⁵ substituents may be the same or different and shall preferably all denote a non-ionic hydrophilic moiety comprising esters, amides and amine moieties, optionally further substituted by a straight chain or branched chain C₁₋₁₀ alkyl groups, preferably C₁₋₅ alkyl groups, where the alkyl groups also may have one or more CH₂ or CH moieties replaced by oxygen or nitrogen atoms. The R⁵ substituents may also further contain one or more groups selected from oxo, hydroxyl, amino or carboxyl derivative, and oxo substituted sulphur and phosphorus atoms. Each of the straight or branched alkyl groups preferably contains 1 to 6 hydroxy groups and more preferably 1 to 3 hydroxy groups. Therefore, in a further preferred aspect, the R⁵ substituents are the same or different and are polyhydroxy C₁₋₅ alkyl, hydroxyalkoxyalkyl with 1 to 5 carbon atoms and hydroxypolyalkoxyalkyl with 1 to 5 carbon atoms, and are attached to the iodinated phenyl group via an amide or a carbamoyl linkage.

The R⁵ groups of the formulas listed below are particularly preferred:

### —CONH—CH₂—CH₂—OH

### —CONN—CH₂—CHOH—CH₂—OH

### —CON(CH₃)CH₂—CHOH—CH₂OH

### —CONH—CH—(CH₂—OH)₂

### —CON—(CH₂—CH₂—OH)₂

### —CON—(CH₂—CHOH—CH₂—OH)₂

### —CONH₂

### —CONHCH₃

### —CONH—CH₂—CH₂—O—CH₃

### —CONH—O—CH₃

### —CONH—CH₂—CHOH—CH₂—O—CH₃

### —CONH—CH₂—CHOCH₃—CH₂—OH

### —CON(CH₂—CHOH—CH₂—OH)(CH₂—CH₂—OH)

### —CONH—C(CH₂—OH)₂CH₃

### —CONH—C(CH₂—OH)₃

### —CONH—CH(CH₂—OH)(CHOH—CH₂—OH)

### —N(COCH₃)H

—N(COCH₃)C₁₋₃alkyl
—N(COCH₃)—mono, bis or tris-hydroxy C₁₋₄ alkyl
—N(COCH₂OH)—hydrogen, mono, bis or tris-hydroxy C₁₋₄ alkyl
—N(CO—CHOH—CH₂OH)—hydrogen, mono, bis or trihydroxylated C₁₋₄ alkyl
—N(CO—CHOH—CHOH—CH₂OH)—hydrogen, mono, bis or trihydroxylated C₁₋₄ alkyl
—N(CO—CH—(CH₂OH)₂)—hydrogen, mono, bis or trihydroxylated C₁₋₄ alkyl, and

### —N(COCH₂OH)₂.

Even more preferably the R⁵ groups will be equal or different and denote one or more moieties of the formulas —CONH—CH₂—CHOH—CH₂—OH, —CON(CH₃)CH₂—CHOH—CH₂OH, —CONH—CH—(CH₂—OH)₂, —CON—(CH₂—CH₂—OH)₂, —CON—(CH₂—CHOH—CH₂—OH)₂, —NH—COCH₂OH, —NH—CO—CHOH—CH₂OH, —NH—CO—CHOH—CHOH—CH₂OH and —N(COCH₂OH)—mono, bis or tris-hydroxy C₁₋₄ alkyl, and even more preferably all R groups are the same and the R⁵ groups in each R are different and denote —CONH—CH₂—CHOH—CH₂—OH, —CON(CH₃)CH₂—CHOH—CH₂OH, —CON—(CH₂—CHOH—CH₂—OH)₂, —NH—CO—CHOH—CH₂OH, —NH—CO—CHOH—CHOH—CH₂OH and —NHCOCH₂OH.

Thus, preferred structures according to the invention include the compounds of formula (II):

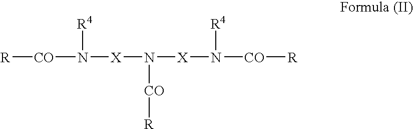

In formula (II), each group R have the meaning above, more preferably at least two of the iodophenyl groups R are the same, and even more preferred all the groups R are the same, and the R⁵ groups all denote non-ionic hydrophilic moieties. The group R⁴ denotes a hydrogen atom or a methyl group, most preferably a hydrogen atom. Each X are the same or different and denotes ethylene or n-propylene and most preferably the X groups are the same and denote ethylene or n-propylene. Most preferably the substituents X and R⁴ in formula (II) are the same.

Some preferred examples the structures according to the invention include the compounds of formulas (IIIa) to (IIIi) below.

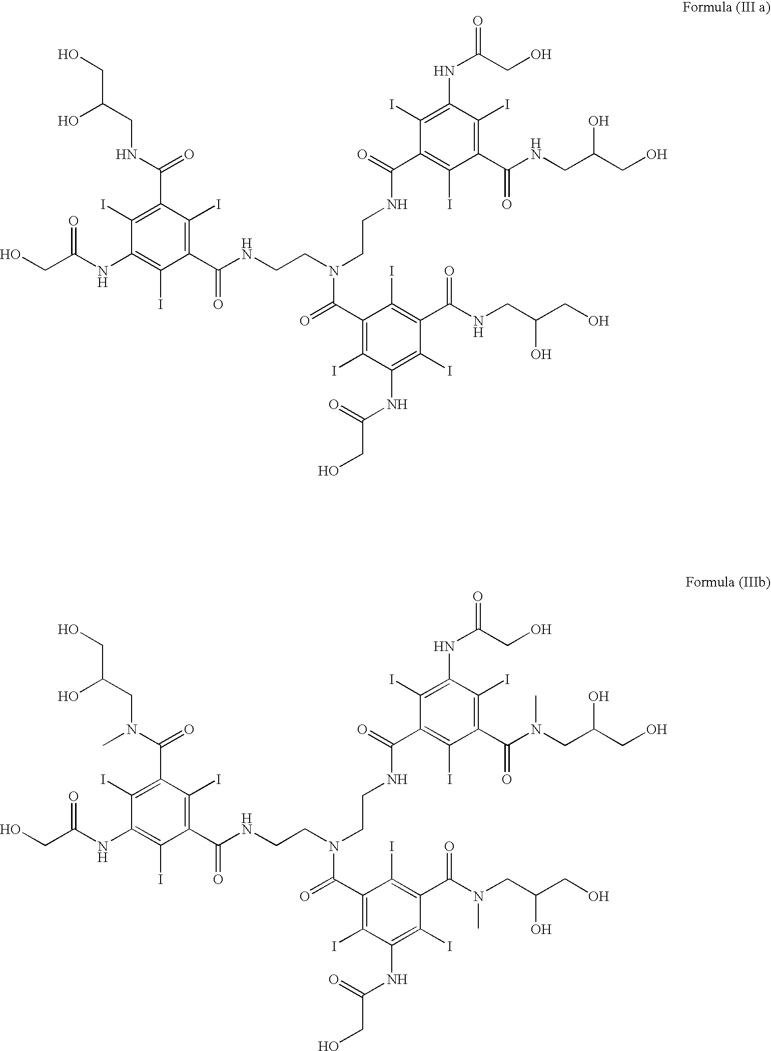

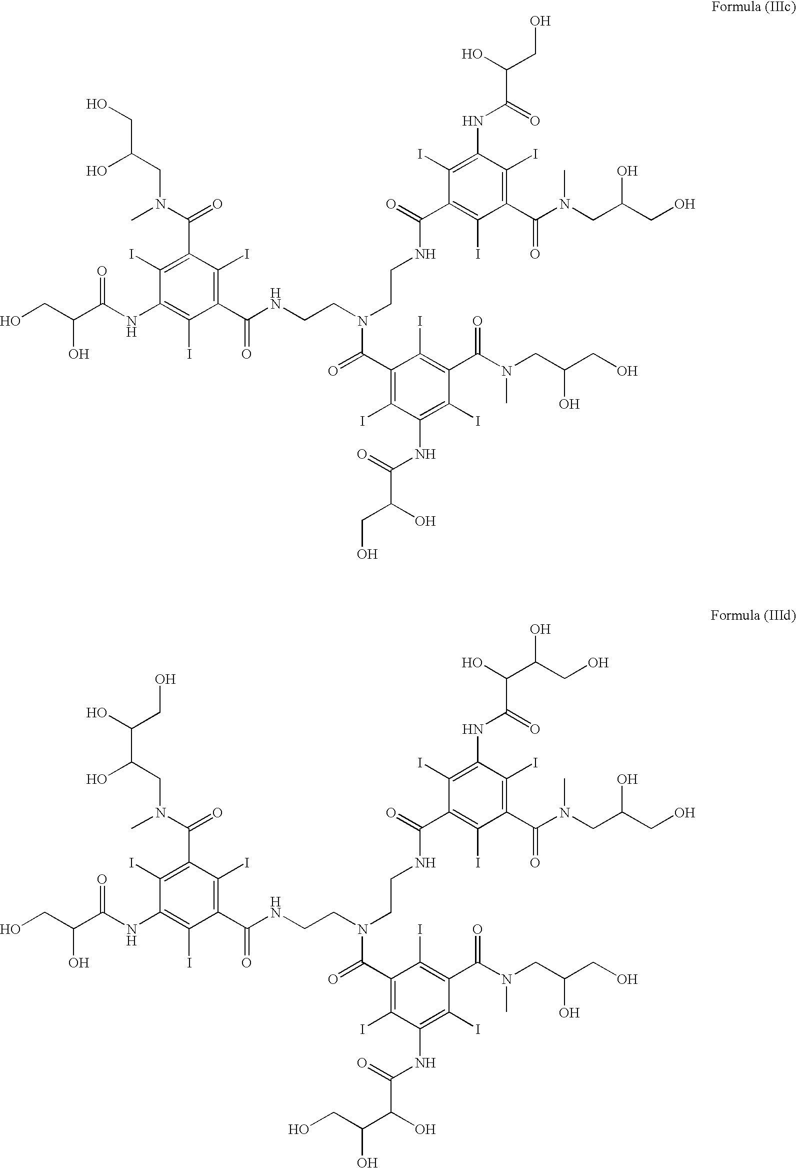

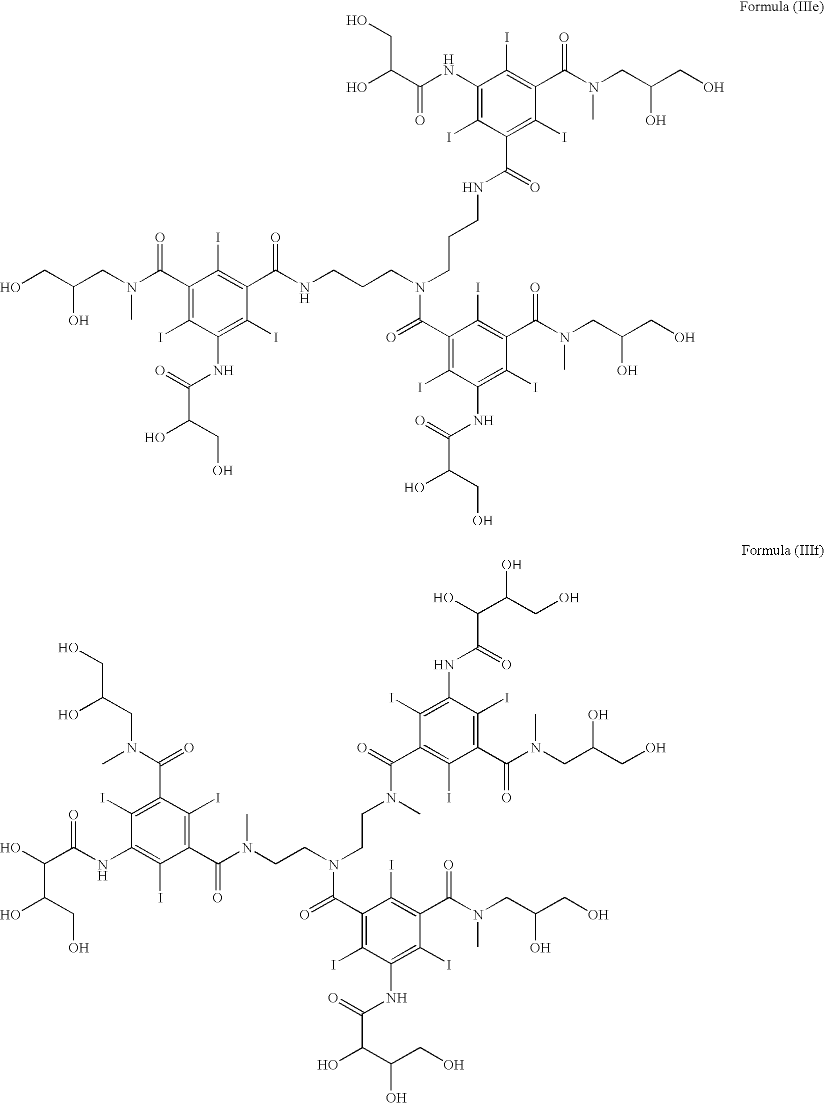

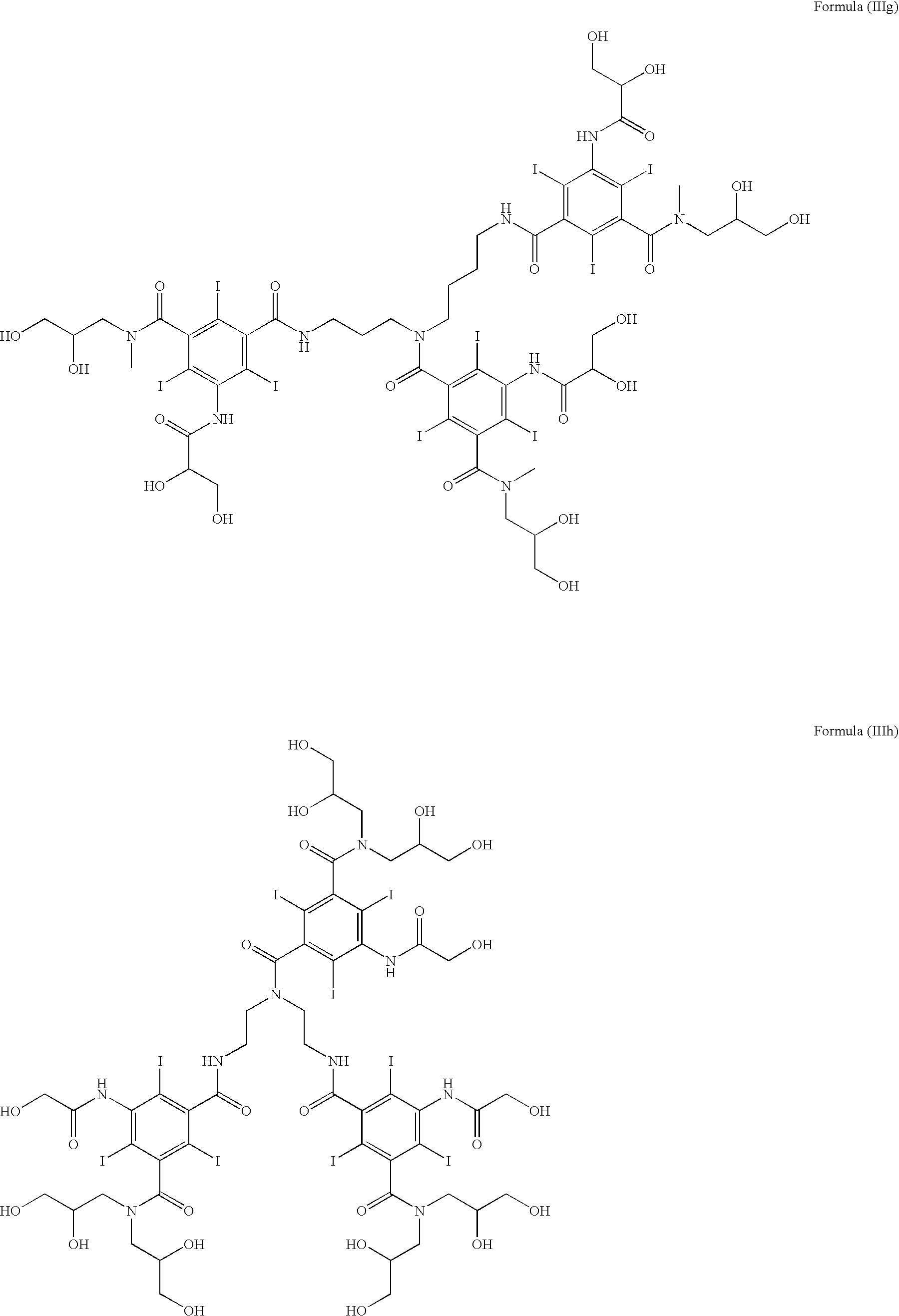

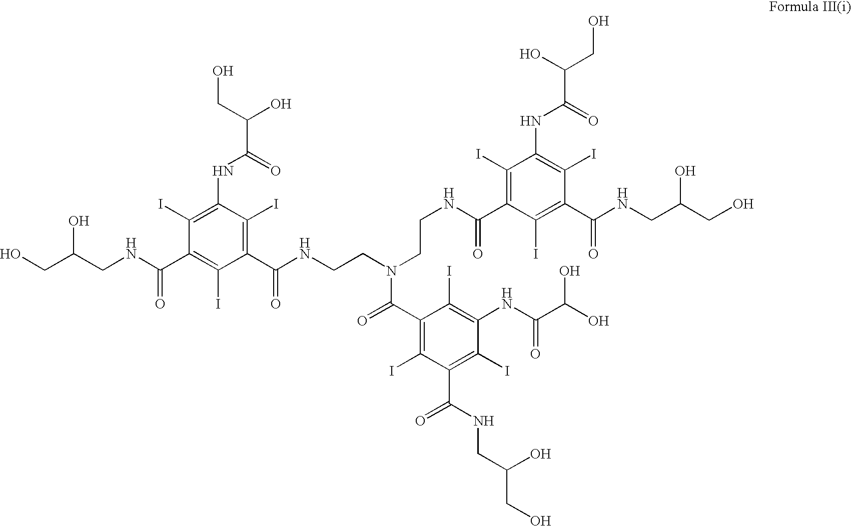

The compounds of formula (I) will attain a relatively compact, folded conformation. Such conformation are relatively round and globular forms such as a star-form with the relatively bulky iodinated phenyl substituents filling up the area between the 3 arms of the star or a “stacked spoon” form where the iodinated phenyl groups are aligned as the spoon “bowls” in a stack of spoons. Globular molecules will usually have enhanced solubility compared with similar molecules with a more planar structure and also have lower viscosities.

At an iodine concentration of 320 mg/ml, which is a common concentration for commercially available iodinated contrast media, the concentration of the compound of formula (I) will be approximately 0.28 M (Molar). The contrast medium will also be hypoosmolar at this iodine concentration, and this is an advantageous property with regards to the nephrotoxicity of the contrast medium. It is also possible to add electrolytes to the contrast medium to lower the cardiovascular effects as explained in WO 90/01194 and WO 91/13636.

Compounds of formula (I) also comprises optical active isomers. Both enantiomerically pure products as well as mixtures of optical isomers are included.

The compounds of the invention may be used as contrast agents and may be formulated with conventional carriers and excipients to produce diagnostic contrast media.

Thus viewed from a further aspect the invention provides a diagnostic composition comprising a compound of formula (I) as described above together with at least one physiologically tolerable carrier or excipient, e.g. in aqueous solution for injection optionally together with added plasma ions or dissolved oxygen.

The contrast agent composition of the invention may be in a ready to use concentration or may be a concentrate form for dilution prior to administration. Generally compositions in a ready to use form will have iodine concentrations of at least 100 mg l/ml, preferably at least 150 mg l/ml, with concentrations of at least 300 mg l/ml, e.g. 320 mg l/ml being preferred. The higher the iodine concentration, the higher is the diagnostic value in the form of X-ray attenuation of the contrast media. However, the higher the iodine concentration the higher is the viscosity and the osmolality of the composition. Normally the maximum iodine concentration for a given contrast media will be determined by the solubility of the contrast enhancing agent, e.g. the iodinated compound, and the tolerable limits for viscosity and osmolality.

For contrast media which are administered by injection or infusion, the desired upper limit for the solution's viscosity at ambient temperature (20° C.) is about 30 mPas, however viscosities of up to 50 to 60 mPas and even more than 60 mPas can be tolerated. For contrast media given by bolus injection, e.g. in angiographic procedures, osmotoxic effects must be considered and preferably the osmolality should be below 1 Osm/kg H₂O, preferably below 850 mOsm/kg H₂O and more preferably about 300 mOsm/kg H₂O.

With the compounds of the invention such viscosity, osmolality and iodine concentrations targets can be met. Indeed, effective iodine concentrations can be reached with hypotonic solutions. It may thus be desirable to make up the solution's tonicity by the addition of plasma cations so as to reduce the toxicity contribution that derives from the imbalance effects following bolus injection. Such cations will desirably be included in the ranges suggested in WO 90/01194 and WO 91/13636.

In particular, addition of sodium and calcium ions to provide a contrast medium isotonic with blood for all iodine concentrations is desirable and obtainable. The plasma cations may be provided in the form of salts with physiologically tolerable counterions, e.g. chloride, sulphate, phosphate, hydrogen carbonate etc., with plasma anions preferably being used.

In a further embodiment the invention provides diagnostic agents comprising a compound of formula (I) and diagnostic compositions comprising a compound of formula (I) together with pharmaceutically acceptable carriers or excipients. The diagnostic agents and composition are preferably for use in X-ray diagnosis.

The contrast media containing compounds of formula (I) can be administered by injection or infusion, e.g. by intervascular administration. Alternatively, contrast media containing compounds of formula (I) may also be administered orally. For oral administration the contrast medium may be in the form of a capsule, tablet or as liquid solution.

Hence, the invention further embraces use of a diagnostic agent and a diagnostic composition containing a compound of formula (I) in X-ray contrast examinations and use of a compound of formula (I) for the manufacture of a diagnostic composition for use as an X-ray contrast agent.

A method of diagnosis comprising administration of compounds of formula (I) to the human or animal body, examining the body with a diagnostic device and compiling data from the examination is also provided. In the method of diagnosis the body may also be preadministrated with compounds of formula (I).

Furthermore, a method of imaging, specifically X-ray imaging is provided, which comprises administration of compounds of formula (I) to the human or animal body, examining the body with a diagnostic device and compiling data from the examination and optionally analysing the data. In the method of imaging the body may also be preadministrated with compounds of formula (I).

### PREPARATION

The compounds of the general formula (I) can be synthesized by multistep procedures from starting materials that are either known from the state of art, that are commercially available or that can be prepared from commercially available starting materials.

Tri-iodinated phenyl groups R and precursors thereof are commercially available or can be produced following procedures described or referred to e.g. in WO95/35122 and WO98/52911. 5-amino-2,4,6-triiodo-isophthalic acid for example is available e.g. from Aldrich.

To synthesize compounds of formula (I), the R⁵ groups or precursors or protected derivatives thereof on the R group are protected if necessary and a reactive compound of R is formed, that is brought to react with an alkyl-triamine of formula (IV):

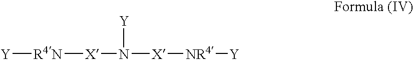

wherein each X′ are as defined for X above or a protected derivative thereof, each R^{4′} are as defined for R⁴ above or a protected derivative thereof above, and each Y are the same or different and denote a leaving group.

Suitable, the reactive functionality on the R-group can be a group containing an acid chloride function. The R⁵ precursor groups or protected groups can suitably be transformed to the R⁵ group or deprotected after the trimeric product is formed. The procedure involves the following steps:

1) functionalization of the iodinated isophthalic amine compound starting material's carboxylic acid groups into acid chlorides as intermediates using traditional methods such as convertion into the corresponding di-acid chloride by treatment with a solution of thionyl chloride in dichloroethane and pyridine according to the procedure described in E. R Marinelli, Tetrahedron, 52, 34, 11177-11214, and as illustrated in Preparation A below.

2) the compound from step 1) is N-acylated followed by the preparation of N-acyl monoamide compounds as illustrated in Preparations B and C below.

3) the compound from step 2) is reacted with an alkyl triamine of formula (IV) as illustrated in the Examples below to form trimers of formula (I). If necessary, hydrolysis of transformation of R^{5′} precursors or protected groups is performed.

The final product is then purified by conventional methods such as semi-preparative HPLC.

### Preparation of Intermediates (A) to (D)

### Preparation (A)

### Synthesis of 5-Amino-2,4,6-triiodo-isophthaloyl dichloride (1)

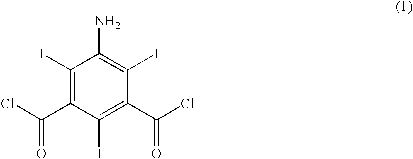

5-Amino-2,4,6-triiodo-isophthalic acid (30 g, 0.054 mol) (commercially available from Aldrich), thionyl chloride (8.2 ml, 0.113 mol) and pyridine (0.2 ml) in 1,2 dichloroethane (20 ml) were heated to 70° C. A portion of thionyl chloride (15.2 ml, 0.21 mol) was added dropwise during 1½ to 2 hrs, and the mixture was heated to 85° C. for 6 hrs. After cooling the reaction mixture to room temperature, it was poured into 300 g of ice-water. The yellow precipitate that formed was filtered off, air dried and then washed with water until washings showed a pH of ca 5. The filter cake was then dried in a vacuum oven at 50° C. for 3 hrs. A light yellow powder was obtained 31 g (˜quant.) as the desired product.

¹³C NMR (DMSOd₆) 66, 78.4, 148.9, 149.2, 169

MS (ES−) found 593.5 [M−H+], expected 593.7

### Preparation B

### N-acylation

### Acetic acid (3,5-bis-chlorocarbonyl-2,4,6-triiodo-phenylcarbamoyl)-methyl ester

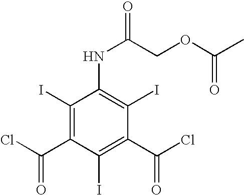

5-Amino-2,4,6-triiodo-isophthaloyl dichloride was dissolved in N,N-dimethylacetamide (DAMc) and a solution of acetoxyacetylchloride (2 eq) in DMAc was slowly added with efficient stirring. The reaction mixture was stirred overnight and the following day, the mixture was slowly poured into stirred ice water. The precipitate was filtered off and dried to give the desired material. The structure was confirmed by ¹H NMR (CDCl₃, 300 MHz): 10.43 (br s, 1H); 4.71 (s, 2H); 2.11 (s, 3H)

Following this procedure a variety of compounds of acylated derivatives can be prepared including:

### Acetic acid 2-acetoxy-1-(3,5-bis-chlorocarbonyl-2,4,6-triiodo-phenylcarbamoyl)-ethyl ester

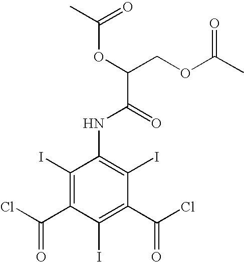

The structure was confirmed by ¹H NMR (CDCl₃, 300 MHz): 10.45 (br s, 1H); 4.49-4.30 (m, 3H); 2.13 (s, 3H).

### Acetic acid 2,3-diacetoxy-1-(3,5-bis-chlorocarbonyl-2,4,6-triiodo-phenylcarbamoyl)propyl ester

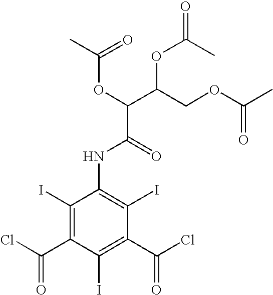

### Preparation C

### Preparation of N-acyl mono-amide derivatives

### Acetic acid {3-chlorocarbonyl-5-[(2,3-dihydroxy-propyl)-methyl-carbamoyl]-2,4,6-triiodo-phenylcarbamoyl}-methyl ester

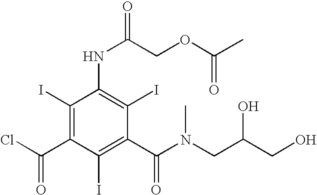

The bis-acid chloride from the previous step was dissolved in DMAc in a dry flask under a nitrogen atmosphere. Triethylamine (2 eq) was added to the solution immediately followed by the addition of 3-methylaminopropane-1,2-diol (2 eq). After stirring overnight, the reaction mixture was concentrated to dryness, and the residue purified by chromatography using silica gel to give the desired product. The structure was confirmed by ¹H NMR (DMSO-D6, 300 MHz): 10.4 (br s, 1H); 4.70 (s, 2H); 3.89-3.83 (m, 1H); 3.75-3.67 (m, 1H); 3.51-3.42 (m, 2H); 3.25-3.15 (m, 1H); 2.85 (s, 3H); 2.15 (s, 3H)

Following this procedure a variety of compounds of formula (IV) can be prepared including:

### Acetic acid 2-acetoxy-1-{3-chlorocarbonyl-5-[(2,3-dihydroxy-propyl)-methylcarbamoyl]-2,4,6-triiodo-phenylcarbamoyl}-ethyl ester

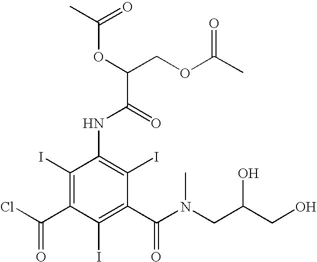

The structure was confirmed by ¹H NMR (DMSO-D6, 300 MHz): 10.4 (br s, 1H); 4.70-4.65 (m, 3H); 3.89-3.83 (m, 1H); 3.75-3.67 (m, 1H); 3.51-3.42 (m, 2H); 3.25-3.15 (m, 1H); 2.85 (s, 3H); 2.15 (s, 3H).

### Acetic acid 2,3-diacetoxy-1-{3-chlorocarbonyl-5-[(2,3-dihydroxy-propyl)-methyl-carbamoyl]-2,4,6-triiodo-phenylcarbamoyl}-propyl ester

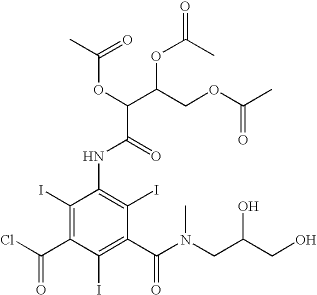

The structure was confirmed by 1H NMR (DMSO-D6, 300 MHz): 10.26-10.37 (br s, 1H); 5.60-5.72 (m, 2H); 4.23-4.35 (m, 1H), 4.03-4.19 (m, 1H); 3.80-4.15 (br, 5H); 3.60-3.80 (m, 1H); 3.13-3.30 (m, 1H); 2.80-2.90 (br s, 3H); 2.26 (s, 3H); 2.08 (s, 3H); 2.02 (s, 3H).

### Acetic acid [3-chlorocarbonyl-5-(2,3-dihydroxy-propylcarbamoyl)-2,4,6-triiodo-phenylcarbamoyl]-methyl ester

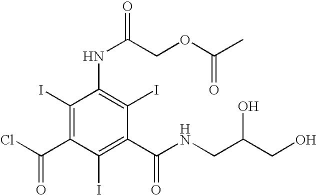

### Acetic acid 2-acetoxy-1-[3-chlorocarbonyl-5-(2,3-dihydroxy-propylcarbamoyl)-2,4,6-triiodo-phenylcarbamoyl]-ethyl ester

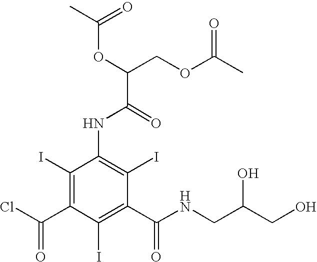

### Acetic acid 2,3-diacetoxy-1-[3-chlorocarbonyl-5-(2,3-dihydroxy-propylcarbamoyl)-2,4,6-triiodo-phenylcarbamoyl]-propyl ester

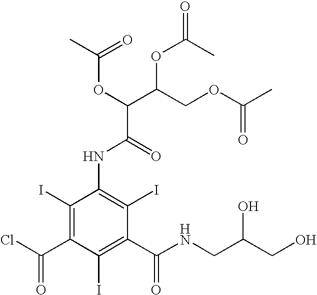

### Acetic acid {3-[bis-(2,3-dihydroxy-propyl)-carbamoyl]-5-chlorocarbonyl-2,4,6-triiodo-phenylcarbamoyl}-methyl ester

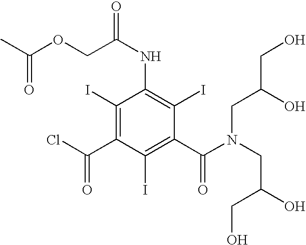

### Acetic acid 2-acetoxy-2-{3-[bis-(2,3-dihydroxy-propyl)-carbamoyl]-5-chlorocarbonyl-2,4,6-triiodo-phenylcarbamoyl}-ethyl ester

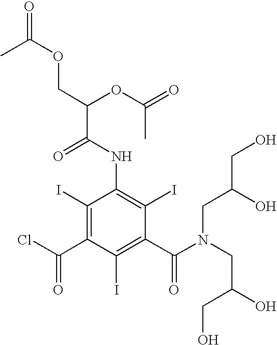

### Acetic acid 2-acetoxy-1-acetoxymethyl-2-{3-[bis-(2,3-dihydroxy-propyl)-carbamoyl]-5-chlorocarbonyl-2,4,6-triiodo-phenylcarbamoyl}-ethyl ester

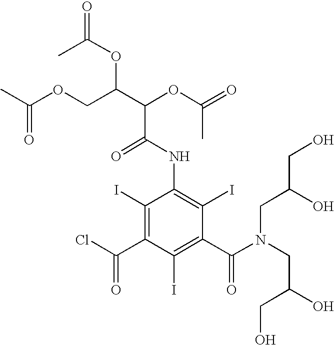

Acetic acid {3-[bis-(2-hydroxy-ethyl)-carbamoyl]-5-chlorocarbonyl-2,4,6-triiodo-phenylcarbamoyl}-methyl ester

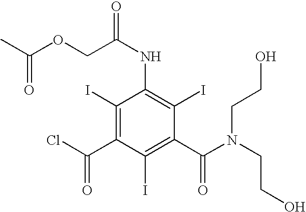

### Acetic acid 2-acetoxy-2-{3-[bis-(2-hydroxy-ethyl)-carbamoyl]-5-chlorocarbonyl-2,4,6-triiodo-phenylcarbamoyl}-ethyl ester

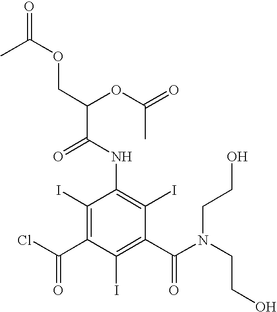

### Acetic acid 2-acetoxy-1-acetoxymethyl-2-{3-[bis-(2-hydroxy-ethyl)-carbamoyl]-5-chlorocarbonyl-2,4,6-triiodo-phenylcarbamoyl}-ethyl ester

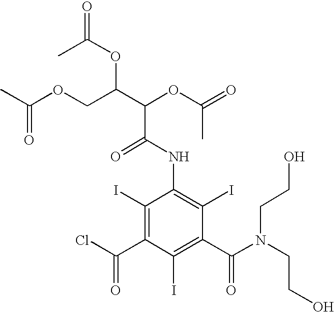

### Acetic acid (3-carbamoyl-5-chlorocarbonyl-2,4,6-triiodo-phenylcarbamoyl)-methyl ester

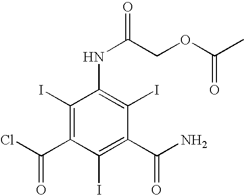

### Acetic acid 2-acetoxy-1-(3-carbamoyl-5-chlorocarbonyl-2,4,6-triiodo-phenylcarbamoyl)-ethyl ester

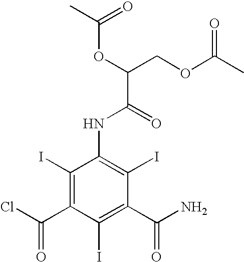

### Acetic acid 2,3-diacetoxy-1-(3-carbamoyl-5-chlorocarbonyl-2,4,6-triiodo-phenylcarbamoyl)-propyl ester

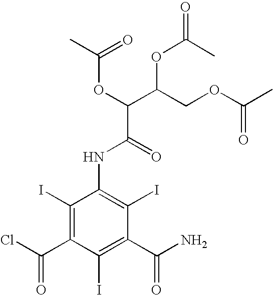

### Preparation D

### Alkyl-Amines

The alkyl-amines are likewise commercially available or readily synthesized from available starting materials.

Examples of alkyl-amine compounds of the formula (IV)

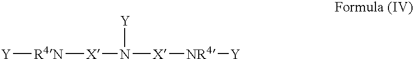

as described above comprise those listed below and those prepared according to the processes in Preparations D1 to D4:

### Diethylenetriamine

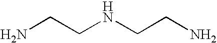

Diethelenetriamine is commercially available from Aldrich

### Dipropylenetriamine

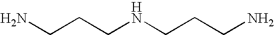

Dipropylenetriamine is commercially available from Aldrich

### N-(2-Amino-ethyl)-propane-1,3-diamine

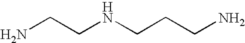

The synthesis of N-(2-Amino-ethyl)-propane-1,3-diamine is described in J. Am. Chem. Soc, 105, 15, 1983, 5002-5011 and references therein.

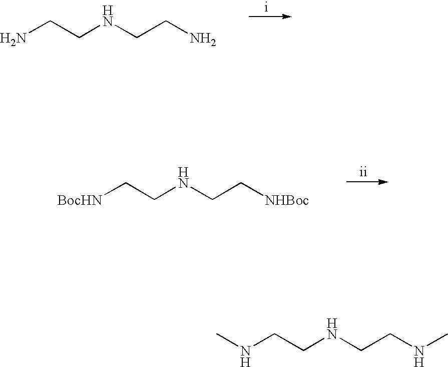

(i) Boc-ON, THF; (ii) LAH, THF

### Preparation D1

### [2-(2-tert-Butoxycarbonylamino-ethylamino)-ethyl]-carbamic acid tert-butyl ester

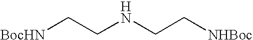

Diethylene triamine was dissolved in anhydrous THF and stirred for 20 min at 0° C. under an atmosphere of nitrogen. A solution of Boc-ON (2.09 eq) in anhydrous THF was slowly added. After complete addition, the reaction mixture was stirred at 0° C. for a further hour. The solvent was then removed under reduced pressure to afford an oil which was purified by chromatography on silica to give the desired product. The structure was confirmed by ¹H NMR (CDCl₃, 300 MHz): 5.46 (br s, 2H); 306-3.01 (m, 4H); 2.55 (t, 4H, J=6 Hz); 1.26 (s, 18H). ¹³C NMR (CDCl₃, 75 MHz): 156.09; 78.71; 48.42; 39.75; 28.08.

Following this procedure a variety of compounds of formula (IV) can be prepared including:

### [3-(3-tert-Butoxycarbonylamino-propylamino)-propyl]-carbamic acid tert-butyl ester

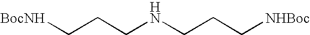

[3-(2-tert-Butoxycarbonylamino-ethylamino)-propyl]-carbamic acid tert-butyl ester

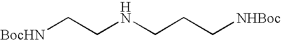

### Preparation D2

### N-Methyl-N′-(2-methylamino-ethyl)-ethane-1,2-diamine

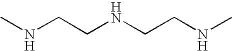

[2-(2-tert-Butoxycarbonylamino-ethylamino)-ethyl]-carbamic acid tert-butyl ester was dissolved into dry THF and slowly added to a suspension of LiAlH₄ in THF. After the complete addition, the mixture was stirred under reflux for 18 h. After cooling the reaction mixture was quenched with wet MgSO₄, and filtered through a pad of celite which was washed several time with THF. The filtrate was then concentrated under reduced pressure to give the desired compound. The structure was confirmed by ¹H NMR (CDCl₃, 300 MHz): 2.78-2.60 (m, 8H); 2.43 (s, 6H).

Following this procedure a variety of compounds of formula (IV) can be prepared including:

### N-Methyl-N′-(3-methylamino-propyl)-propane-1,3-diamine

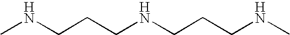

### N-Methyl-N′-(2-methylamino-ethyl)-propane-1,3-diamine

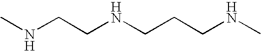

### 1-Amino-3-(2-aminoethylamino)propan-2-ol

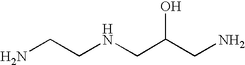

The synthesis of 1-amino-3-(2-aminoethylamino)propan-2-ol is described in Chem. Pharm. Bull., 37, 11, 1989, 3139-3141 and references therein.

### 1-Amino-3-(3-aminopropylamino)propan-2-ol

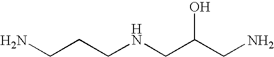

The synthesis of 1-Amino-3-(3-aminopropylamino)propan-2-ol is described in Chem. Pharm. Bull., 37, 11, 1989, 3139-3141 and references therein.

### 1-Amino-3-(3-amino-2-hydroxypropylamino)propan-2-ol

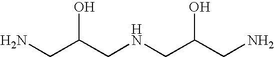

The synthesis of 1-Amino-3-(3-amino-2-hydroxypropylamino)propan-2-ol is the U.S. Pat. No. 2,046,720.

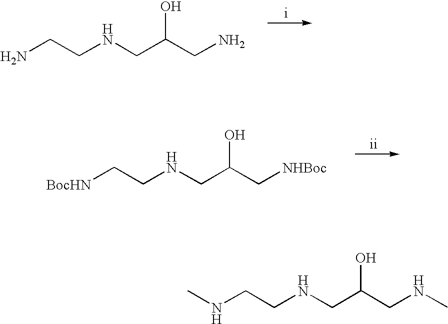

(i) Boc-ON, THF; (ii) LAH, THF

### Preparation D3

### [3-(2-tert-Butoxycarbonylaminoethylamino)-2-hydroxypropyl]carbamic acid tert-butyl ester

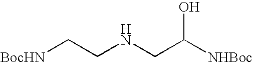

Amino-3-(2-aminoethylamino)propan-2-ol is dissolved in anhydrous THF and stirred at 0° C. under an atmosphere of nitrogen. A solution of Boc-ON (2.09 eq) in anhydrous THF is slowly added. After complete addition, the reaction mixture is stirred at 0° C. After completion, the solvent is removed under reduced pressure. The crude material is purified by chromatography on silica to afford the desired material.

Following this procedure a variety of compounds of formula (IV) can be prepared including:

### [3-(3-tert-Butoxycarbonylamino-propylamino)-2-hydroxy-propyl]-carbamic acid tert-butyl ester

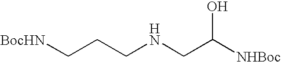

[3-(3-tert-Butoxycarbonylamino-2-hydroxy-propylamino)-2-hydroxy-propyl]-carbamic acid tert-butyl ester

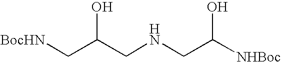

### Preparation D4

### 1-Methylamino-3-(2-methylamino-ethylamino)-propan-2-ol

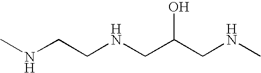

[3-(2-tert-Butoxycarbonylaminoethylamino)-2-hydroxy-propyl-carbamic acid tert-butyl ester is dissolved into dry THF and slowly added to a suspension of LiAlH₄ in THF. After the complete addition, the mixture is stirred under reflux. Whereupon, the reaction mixture is cooled to room temperature, quenched with wet MgSO₄, and filtered through a pad of celite which is washed several time with THF. The filtrate is then concentrated under reduced pressure to give the desired compound.

Following this procedure a variety of compounds of formula (IV) can be prepared including:

### 1-Methylamino-3-(3-methylaminopropylamino)propan-2-ol

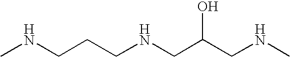

### 1-(2-Hydroxy-3-methylaminopropylamino)-3-methylaminopropan-2-ol

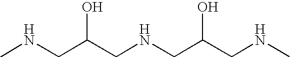

The formation of the trimers of formula (I) are formed by reacting the reactive precursor of the group R with an alkyl-amine compound of formula (IV) as further illustrated in the non-limiting examples 1 to 7 below.

To prepare compounds of formula (I) where the substituents R in formula (I) are different, specific measures have to be taken.

Compounds of formula (IIa)

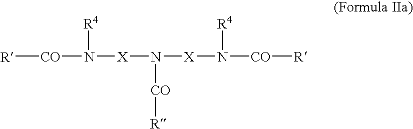

wherein R′ and R″ have the meaning of R but are different from one another, can be prepared by selectively forming a dimer by reacting reactive the acid chlorides R′COCl with a compound of formula (IV) at room temperature. In the second step, an acid chloride of formula R″COCl is reacted with the dimer formed in the first step at elevated temperature, e.g. at about 60° C. As a final step the trimer formed is deprotected. This procedure is exemplified below:

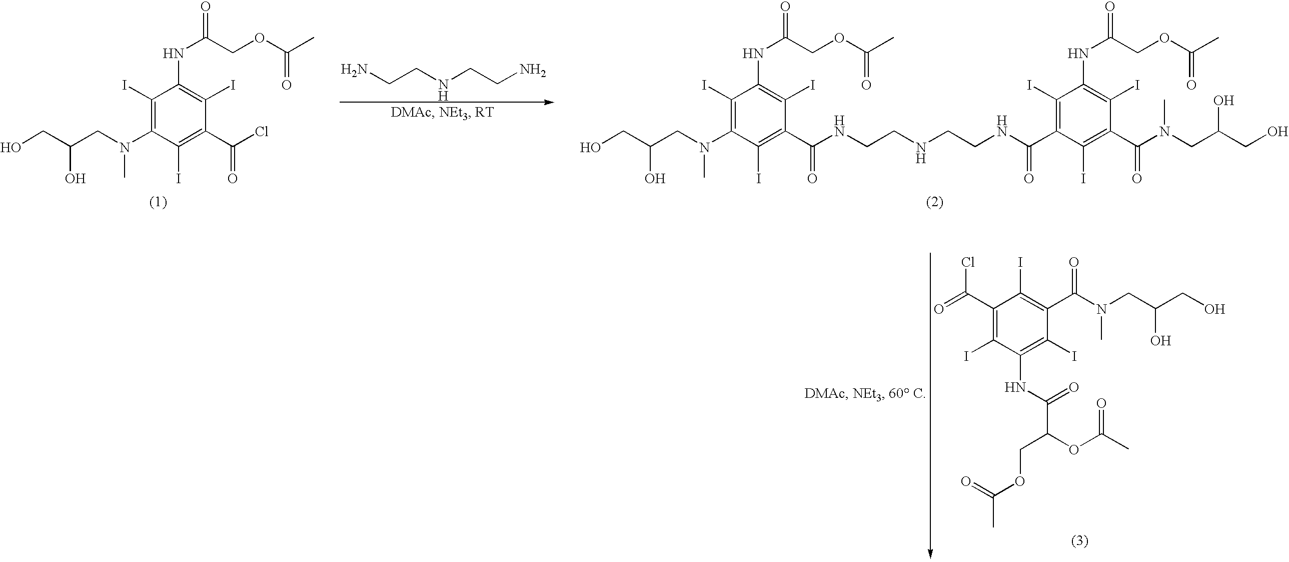

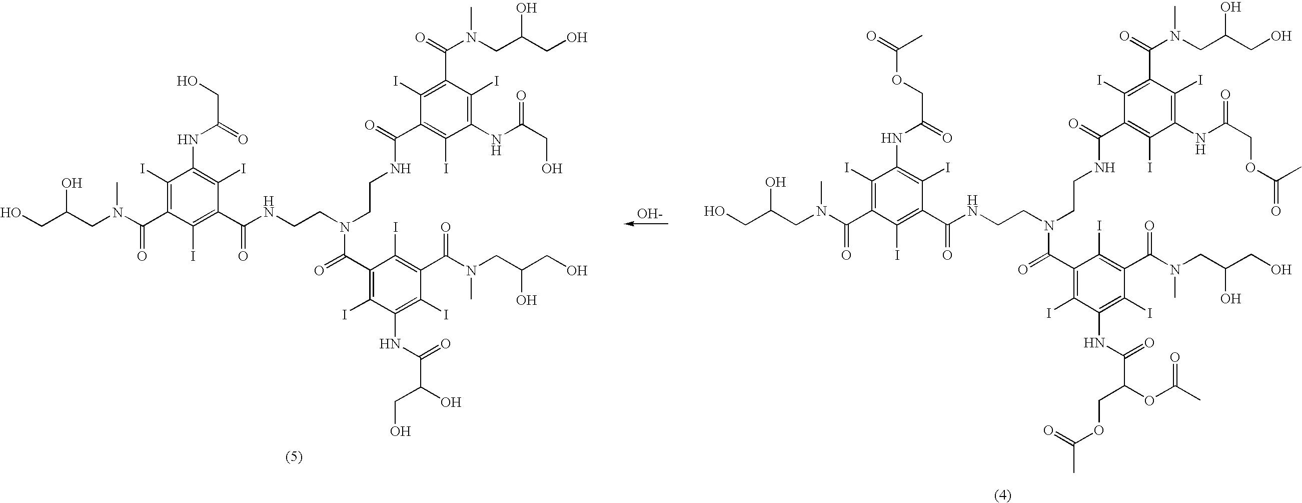

### Acetic acid {3-[2-(2-{3-(2-acetoxy-acetylamino)-5-[(2,3-dihydroxy-propyl)-methyl-amino]-2,4,6-triiodo-benzoylamino}-ethylamino)-ethylcarbamoyl]-5-[(2,3-dihydroxypropyl)-methyl-carbamoyl]-2,4,6-triiodo-phenylcarbamoyl}-methyl ester (2)

Acetic acid {3-chlorocarbonyl-5-[(2,3-dihydroxypropyl)methylcarbamoyl]-2,4,6-triiodophenylcarbamoyly}-methyl ester (1) (7.36 g, 10 mmol) is dissolved in N,N-dimethylacetamide (30 ml) and triethylaine (10 mmol) added. To this solution is added diethylenetriamine (5 mmol) and the solution stirred at 20° C. for 12 h. The reaction is monitored for consumption of all compound (1). Compound (2) can be isolated in pure form by reverse phase chromatography after removal of solvent under reduced pressure.

### 1,5-Bis-{3-[2-(2-{3-(2-acetoxy-acetylamino)-5-[(2,3-dihydroxy-propyl)-methyl-amino]-2,4,6-triiodo-phenylcarbamoyl}-3-{3-[2,3-diacetoxypropanoylamino]-5-[(2,3-dihydroxy-propyl)-methyl-carbamoyl]-2,4,6-triiodo-phenyl}-3-azapentane (4)

Acetic acid 2-acetoxy-1-[3-chlorocarbonyl-5-(2,3-dihydroxypropylcarbamoyl)-2,4,6-triiodo-phenylcarbamoyl]ethyl ester (3) (6 mmol) is added to a solution of compound (2) (5 mmol) in N,N-dimethylacetamide (30 ml) and triethylamine (5 mmol) and the mixture heated at 60° C. for 18 h. The product is isolated by removal of solvent and reverse phase chromatography.

### 1,5-Bis-{3-[(2-(2-{3-(2-hydroxy-acetylamino)-5-[(2,3-dihydroxy-propyl)-methyl-amino]-2,4,6-triiodo-phenylcarbamoyl}-3-{3-[2,3-dihydroxypropanoylamino]-5-[(2,3-dihydroxy-propyl)-methyl-carbamoyl]-2,4,6-triiodo-phenyl}-3-azapentane (5)

Compound (4) is dissolved in methanol water (1:1) containing a small amount of sodium hydroxide. Removal of the acetate groups is monitored by LCMS. On completion the solvent is evaporated and the product purified using preparative HPLC.

Compounds of formula (IIb)

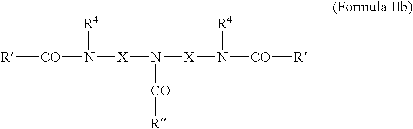

wherein R′, R″ and R′″ have the meaning of R but are different from one another, can be prepared by using a triamine alkyl group of formula (IV) where all Y-groups are different. Examples of such protected triamine are compounds of formula (8) below. Compounds of these structures can be prepared by the chemistry described in Cushman et al J. Med. Chem., 2003, 46, 5712-5724:

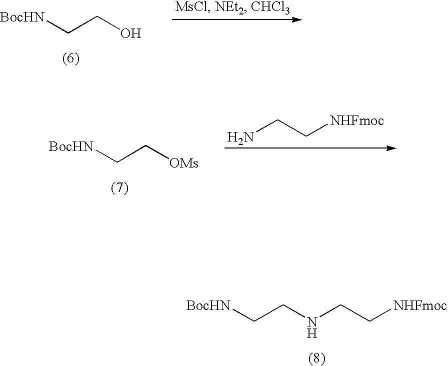

The protected triamine is reacted with reactive an acid chloride, R″COCl to form a monomer. After the deprotection of one of the protecting groups the compound from the first step is reacted with one R′COCl and R′″COCl to form a dimer. Thereafter, the last protection group is removed and the dimer in reacted with the other of R′COCl and R′″COCl to form the trimer followed by full deprotection. As a final step the trimer formed is deprotected. This procedure is exemplified below:

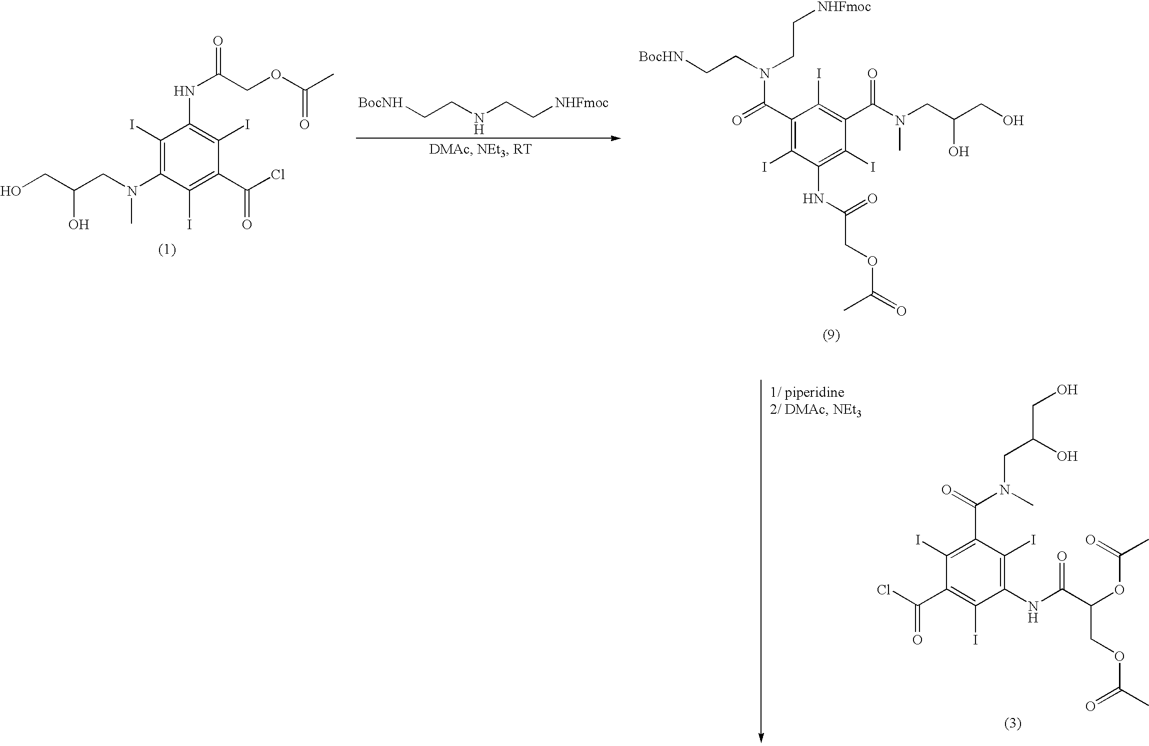

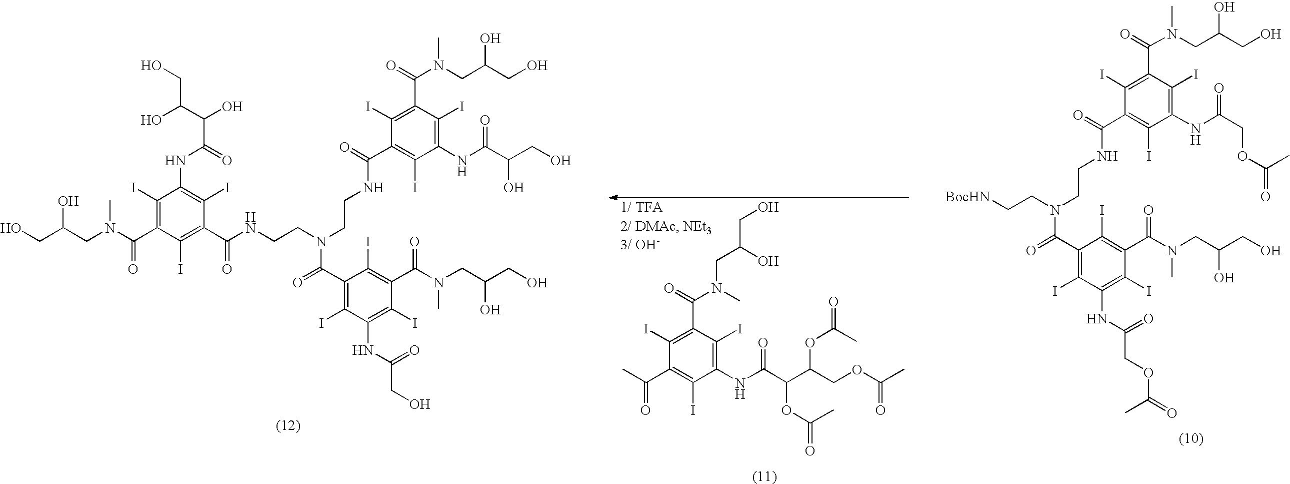

Compounds of formula (IIc)

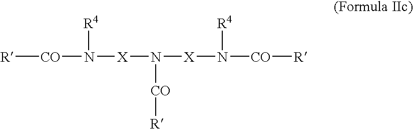

wherein R′, and R″ have the meaning of R but are different from one another, can be prepared by following using a triamine alkyl group of formula (IV) where the Y-groups on the central and one terminal nitrogen are hydrogen atoms and the last terminal nitrogen atom is protected. A dimer is formed by reacting with R′COCl, then deprotecting of the remaining terminal nitrogen by removing the Y group finally reacting the dimer with the compound R″COCl to form the trimer. As a final step the trimer formed is deprotected. This procedure is exemplified below:

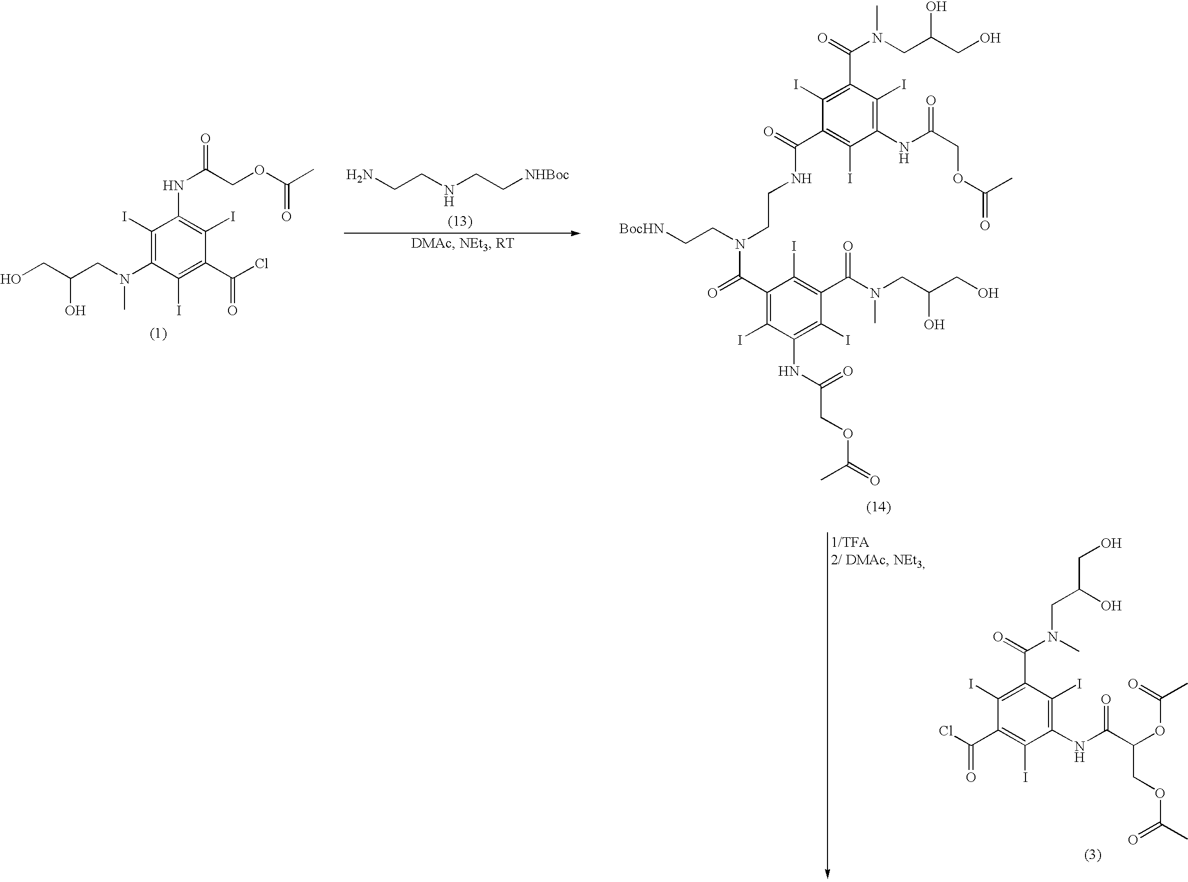

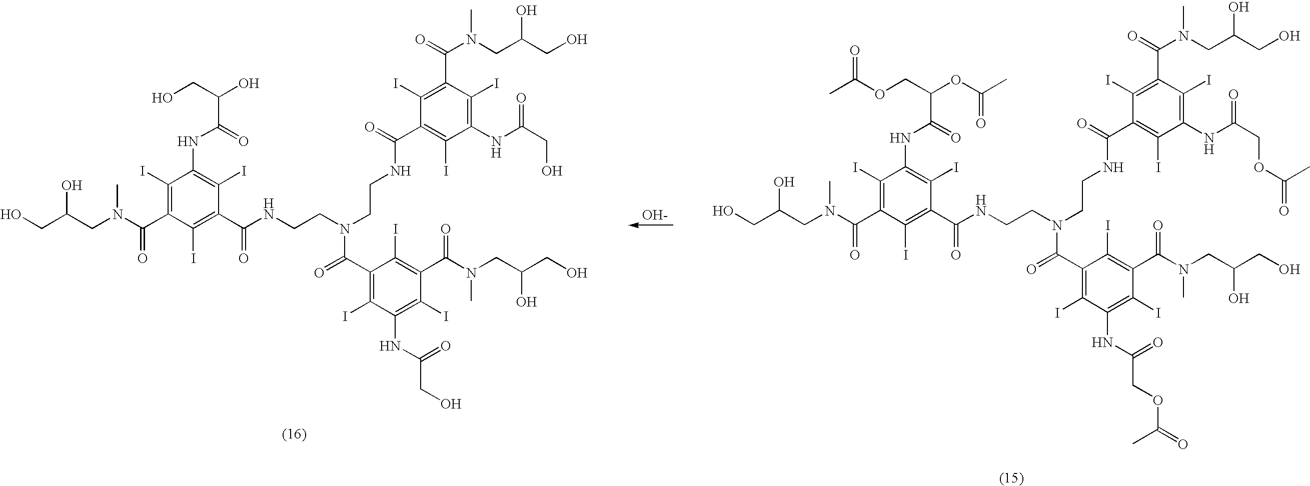

The preparation of compounds of formula (I) will now be illustrated by the following, non-limiting examples:

### Example 1

### N,N-Bis-[2-(N-(2,3-dihydroxy-propyl)-5-(2-hydroxy-acetylamino)-2,4,6-triiodo-isophthalylamino)-ethyl]-N′-(2,3-dihydroxy-propyl)-5-(2-hydroxy-acetylamino)-2,4,6-triiodo-isophthalamide

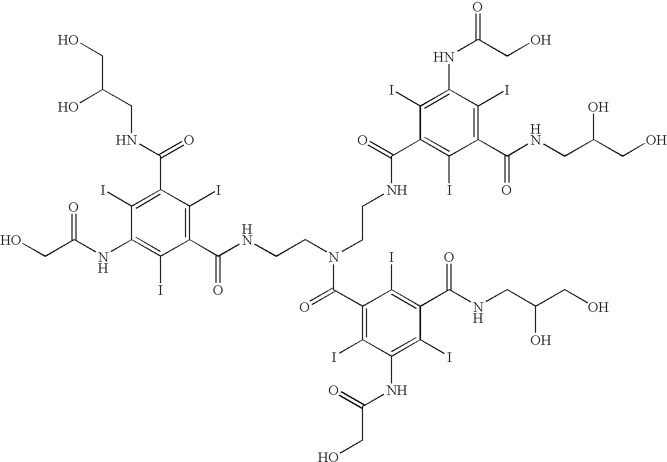

To a solution of acetic acid [3-chlorocarbonyl-5-(2,3-dihydroxypropylcarbamoyl)-2,4,6-triiodophenylcarbamoyl]methyl ester in DMAc was added 0.3 equivalent of diethylenetriamine and 0.3 equivalent of triethylamine. The reaction was stirred at ambient temperature until the reaction proceeds no further. The reaction mixture was extracted into ethyl acetate and washed with water to remove the DMAc. The organic layer was dried over MgSO4 and the filtrate concentrated under vacuum to give the desired compound which was used in the next step without purification. The crude material was dissolved in the minimum amount of methanol and treated with aqueous ammonia. The reaction was stirred at ambient temperature and monitored by LC-MS, whereupon the reaction mixture was concentrated to dryness, dissolved in the minimum amount of water, filtered and purified by preparative HPLC to give the desired final product. The structure was confirmed by LC-MS (1060 [M/2+H] in ES+), HPLC single peak

### Example 2

### N,N-Bis-[2-(N-(2,3-dihydroxy-propyl)-5-(2-hydroxy-acetylamino)-N-methyl-2,4,6-triiodo-isophthalylamino)-ethyl]-N′-(2,3-dihydroxy-propyl)-5-(2-hydroxy-acetylamino)-N′-methyl-2,4,6-triiodo-isophthalamide

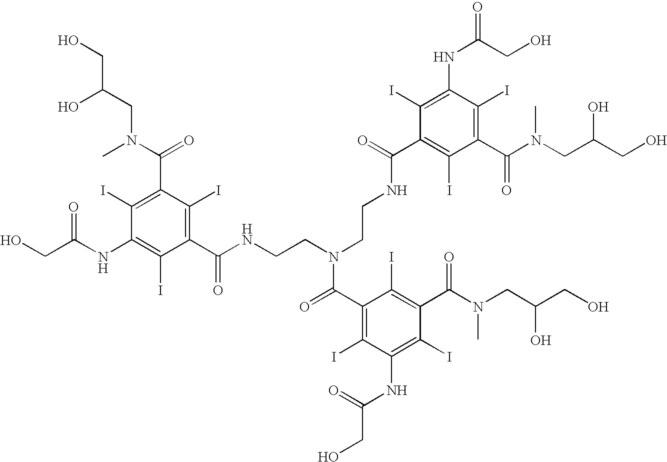

This compound was produced following the procedure of example 1.

The structure was confirmed by LC-MS (1081 [M/2+H] in ES+), HPLC single peak

### Example 3

### N,N-Bis-[2-(N-(2,3-dihydroxy-propyl)-5-(2,3-dihydroxy-propionylamino)-N-methyl-2,4,6-triiodo-isophthalylamino)-ethyl]-N′-(2,3-dihydroxy-propyl)-5-(2,3-dihydroxy-propionylamino)-N′-methyl-2,4,6-triiodo-isophthalamide

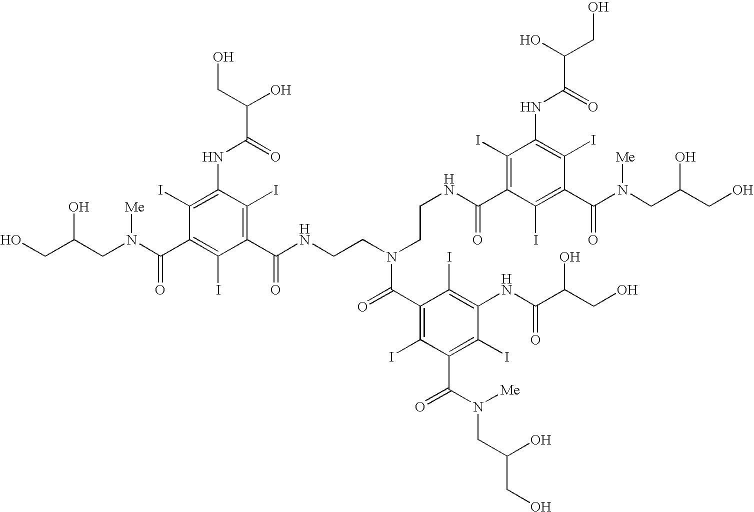

This compound was produced following the procedure of example 1.

The crude product was purified by preparative reverse phase HPLC (C18 Luna column: 10 micron, 250×50 mm), eluting with water (A) and acetonitrile (B) (2% B→20% B) with a flow of 175 mL/min, to afford N,N-Bis-[2-(N-(2,3-dihydroxy-propyl)-5-(2,3-dihydroxy-propionylamino)-N-methyl-2,4,6-triiodo-isophthalylamino)-ethyl]-N′-(2,3-dihydroxy-propyl)-5-(2,3-dihydroxy-propionylamino)-N′-methyl-2,4,6-triiodo-isophthalamide. The structure was confirmed by NMR. LC-MS: calcd for C₄₉H₅₈I₉N₉O₂₁ 2250.5; found, 1124.0 (M/2−H⁻)⁻.

### Example 4

### N,N-Bis-[2-(N-(2,3-dihydroxy-propyl)-N-methyl-5-(2,3,4-trihydroxy-butyrylamino)-2,4,6-triiodo-isophthalylamino)-ethyl]-N′-(2,3-dihydroxy-propyl)-N′-methyl-5-(2,3,4-trihydroxy-butyrylamino)-2,4,6-triiodo-isophthalamide

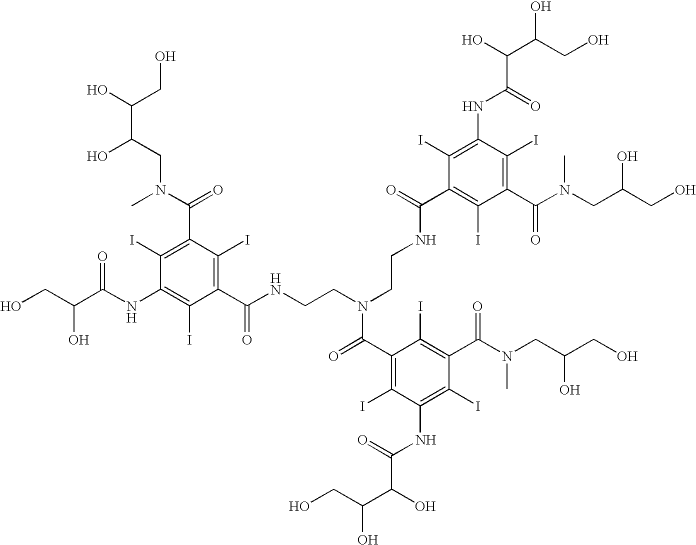

This compound was produced following the procedure of example 1.

The structure was confirmed by LC-MS (1171 [M/2+H] in ES+), HPLC single peak

### Example 5

### N,N-Bis-[2-(N-(2,3-dihydroxy-propyl)-N-methyl-5-(2,3,4-trihydroxy-butyrylamino)-2,4,6-triiodo-isophthalylamino)-2-methylamino-ethyl]-N′-(2,3-dihydroxy-propyl)-N′-methyl-5-(2,3,4-trihydroxy-butyrylamino)-2,4,6-triiodo-isophthalamide

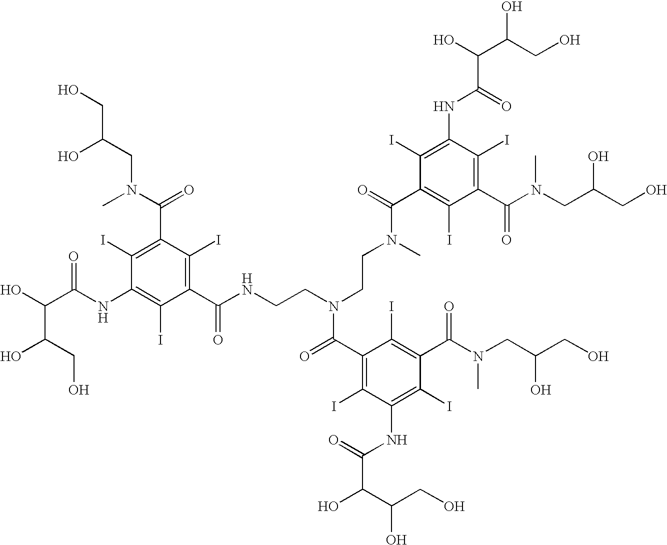

This compound was produced following the procedure of example 1.

The structure was confirmed by UV-chromatogram (DAD, TIC) m/z 2369.5 (MH⁺) and its doubly charged ion at m/z 1185.3

### Example 6

### N,N-Bis-[3-(N-(2,3-dihydroxy-propyl)-5-(2,3-dihydroxy-propionylamino)-N-methyl-2,4,6-triiodo-isophthalylamino)-propyl]-N′-(2,3-dihydroxy-propyl)-5-(2,3-dihydroxy-propionylamino)-N′-methyl-2,4,6-triiodo-isophthalamide

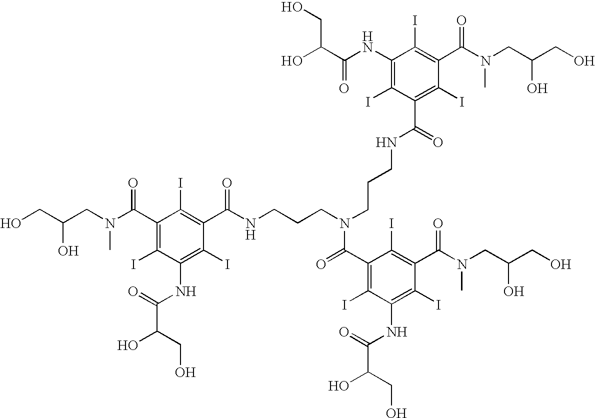

This compound was produced following the procedure of example 1.

The structure was confirmed by UV-chromatogram (DAD, TIC) m/z 2279.5 (MW), and the doubly charged species at m/z 1140.3

### Example 7

### N-[2-(N-(2,3-dihydroxy-propyl)-5-(2,3-dihydroxy-propionylamino)-N-methyl-2,4,6-triiodo-isophthalylamino)-propyl]-N-[3-(N-(2,3-dihydroxy-propyl)-5-(2,3-dihydroxy-propionylamino)-N-methyl-2,4,6-triiodo-isophthalylamino)-butyl]-N′-(2,3-dihydroxy-propyl)-5-(2,3-dihydroxy-propionylamino)-N′-methyl-2,4,6-triiodo-isophthalamide

a) N,N′,N″-Tris[acetic acid 2-acetoxy-1-{5-[(2,3-dihydroxy-propyl)-methyl-carbamoyl]-2,4,6-triiodo-phenylcarbamoylyethyl}-ester]benzoyl]1,5,10-triazadecane

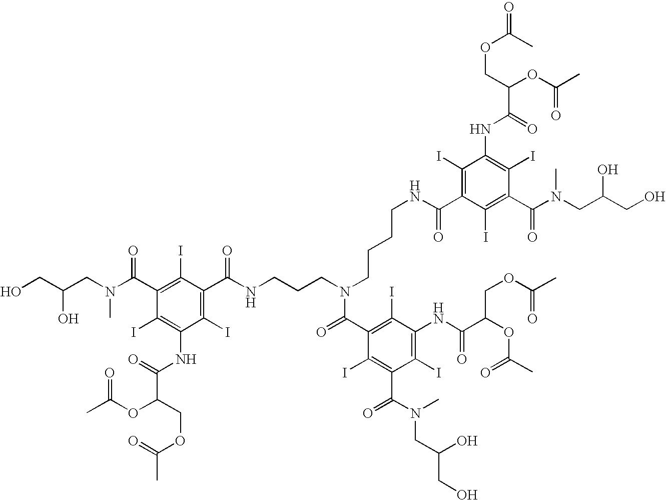

A round bottomed flask was charged with acetic acid 2-acetoxy-1-{3-chlorocarbonyl-5-[(2,3-dihydroxypropyl)methylcarbamoyl]-2,4,6-triiodophenylcarbamoyl}ethyl ester (3.0 g, 3.6 mmol), DMAc (5 mL) and spermidine, (183 mg, 1.2 mmol) and triethylamine (750 mg, 7.5 mmol) under a nitrogen atmosphere at ambient temperature. After 24 hours the reaction mixture was purified by silica gel column chromatography eluting with methanol and DCM. The desired fraction were concentrated and analysed by LCMS. This gave 1.2 g of the desired trimer.

MS (ES+) found 1273.48 [M/2+H]

b) N-[2-(N-(2,3-dihydroxy-propyl)-5-(2,3-dihydroxy-propionylamino)-N-methyl-2,4,6-triiodo-isophthalylamino)-propyl]-N-[3-(N-(2,3-dihydroxy-propyl)-5-(2,3-dihydroxy-propionylamino)-N-methyl-2,4,6-triiodo-isophthalylamino)-butyl]-N′-(2,3-dihydroxy-propyl)-5-(2,3-dihydroxy-propionylamino)-N′-methyl-2,4,6-triiodo-isophthalamide

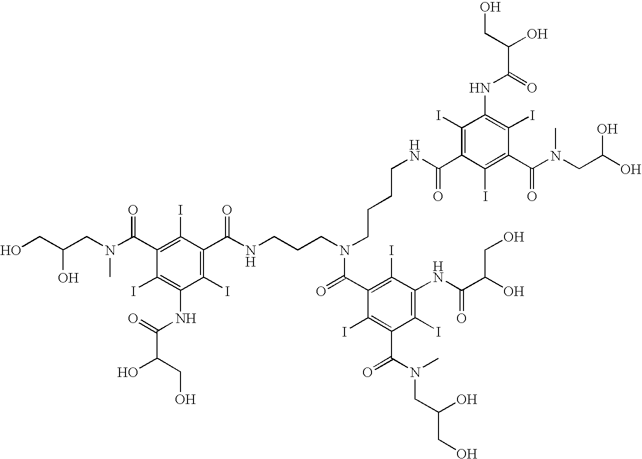

A flask was charged with N,N′,N″-Tris[acetic acid 2-acetoxy-1-{5-[(2,3-dihydroxy-propyl)-methyl-carbamoyl]-2,4,6-triiodo-phenylcarbamoyl}-ethyl ester]benzoyl]1,5,10-triazadecane (1 g), and methanol (5 mL). To this was added sodium methoxide (25 mg) in one portion. The mixture was stirred at ambient temperature. After 24 hours the mixture was diluted with water (5 mL) and the crude mixture was separated by preparative HPLC. The desired fractions were concentrated and freeze dried to yield a white powder. The purity was confirmed by LCMS.

MS (ES+) 2293.5 (MH⁺)

Following the procedure above the following compounds can also producible:

a) N,N-Bis-[2-(N-(2,3-dihydroxy-propyl)-5-(2,3-dihydroxy-propionylamino)-2,4,6-triiodo-isophthalylamino)-ethyl]-N′-(2,3-dihydroxy-propyl)-5-(2,3-dihydroxy-propionylamino)-2,4,6-triiodo-isophthalamide

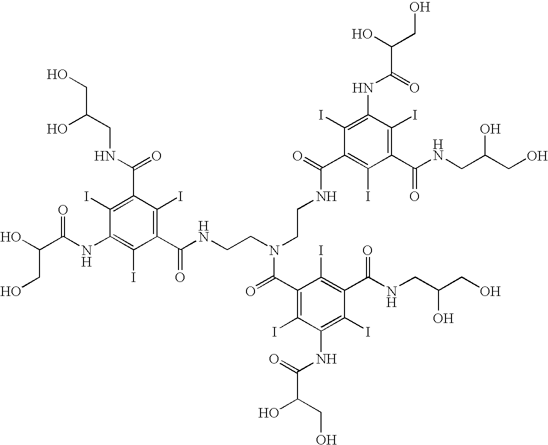

b) N,N-Bis-[2-(N-(2,3-dihydroxy-propyl)-5-(2,3,4-trihydroxy-butyrylamino)-2,4,6-triiodo-isophthalylamino)-ethyl]-N′-(2,3-dihydroxy-propyl)-5-(2,3,4-trihydroxy-butyrylamino)-2,4,6-triiodo-isophthalamide

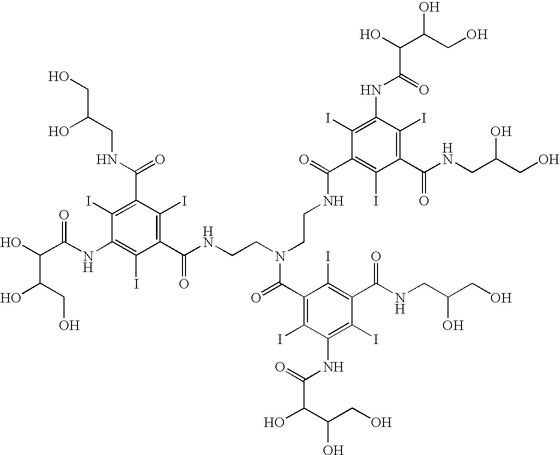

c) N,N-Bis-[2-(N-(2,3-dihydroxy-propyl)-5-(2-hydroxy-acetylamino)-2,4,6-triiodo-isophthalylamino)-2-methylamino-ethyl]-N′-(2,3-dihydroxy-propyl)-5-(2-hydroxy-acetylamino)-2,4,6-triiodo-isophthalamide

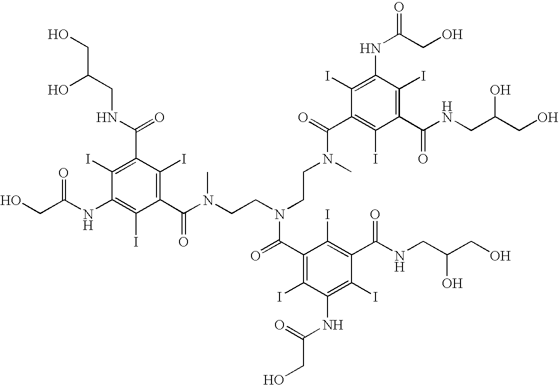

d) N,N-Bis-[2-(N-(2,3-dihydroxy-propyl)-5-(2,3-dihydroxy-propionylamino)-2,4,6-triiodo-isophthalylamino)-2-methylamino-ethyl]-N′-(2,3-dihydroxy-propyl)-5-(2,3-dihydroxy-propionylamino)-2,4,6-triiodo-isophthalamide

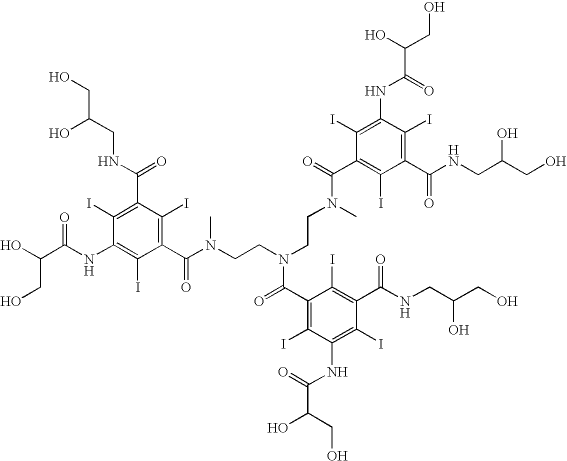

e) N,N-Bis-[2-(N-(2,3-dihydroxy-propyl)-5-(2,3,4-trihydroxy-butyrylamino)-2,4,6-triiodo-isophthalylamino)-2-methylamino-ethyl]-N′-(2,3-dihydroxy-propyl)-5-(2,3,4-trihydroxy-butyrylamino)-2,4,6-triiodo-isophthalamide

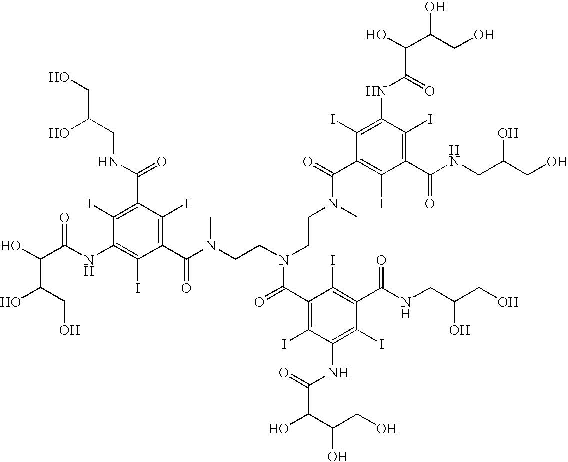

f) N,N-Bis-[2-(N-(2,3-dihydroxy-propyl)-5-(2-hydroxy-acetylamino)-N-methyl-2,4,6-triiodo-isophthalylamino)-2-methylamino-ethyl]-N′-(2,3-dihydroxy-propyl)-5-(2-hydroxy-acetylamino)-N′-methyl-2,4,6-triiodo-isophthalamide

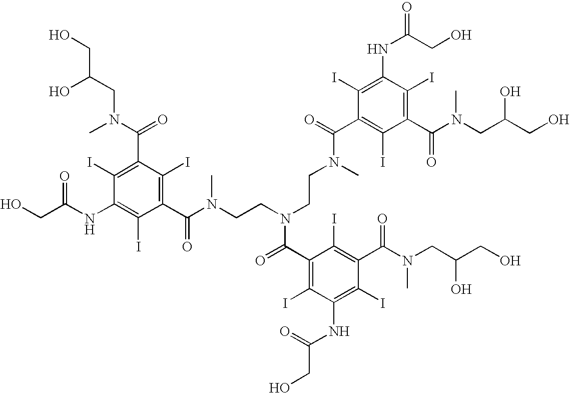

q) N,N-Bis-[2-(N-(2,3-dihydroxy-propyl)-5-(2,3-dihydroxy-propionylamino)-N-methyl-2,4,6-triiodo-isophthalylamino)-2-methylamino-ethyl]-N′-(2,3-dihydroxy-propyl)-5-(2,3-dihydroxy-propionylamino)-N′-methyl-2,4,6-triiodo-isophthalamide

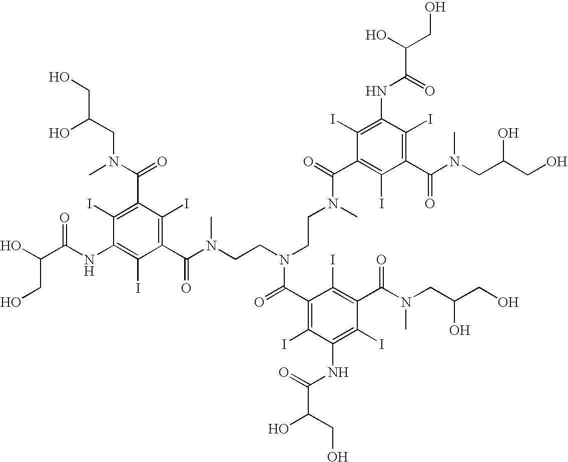

h) N,N-Bis-[3-(N-(2,3-dihydroxy-propyl)-5-(2-hydroxy-acetylamino)-2,4,6-triiodo-isophthalylamino)-propyl]-N′-(2,3-dihydroxy-propyl)-5-(2-hydroxy-acetylamino)-2,4,6-triiodo-isophthalamide

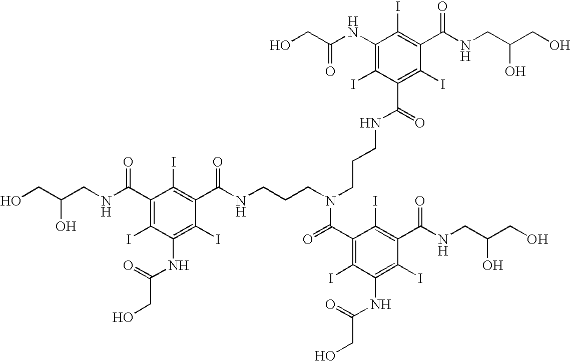

i) N,N-Bis-[3-(N-(2,3-dihydroxy-propyl)-5-(2,3-dihydroxy-propionylamino)-2,4,6-triiodo-isophthalylamino)-propyl]-N′-(2,3-dihydroxy-propyl)-5-(2,3-dihydroxy-propionylamino)-2,4,6-triiodo-isophthalamide

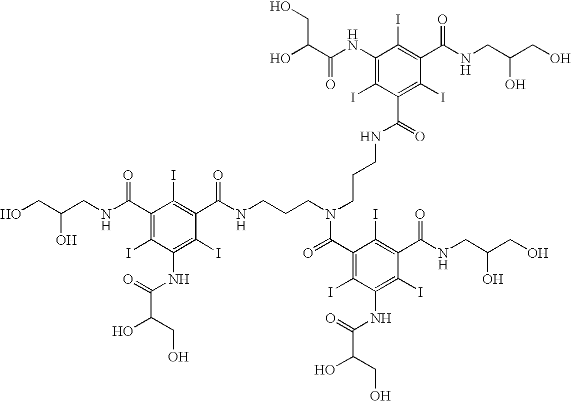

j) N,N-Bis-[3-(N-(2,3-dihydroxy-propyl)-5-(2,3,4-trihydroxy-butyrylamino)-2,4,6-triiodo-isophthalylamino)-propyl]-N′-(2,3-dihydroxy-propyl)-5-(2,3,4-trihydroxy-butyrylamino)-2,4,6-triiodo-isophthalamide

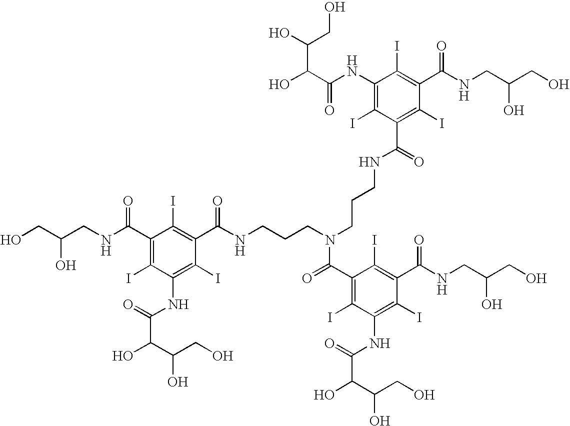

k) N,N-Bis-[3-(N-(2,3-dihydroxy-propyl)-5-(2-hydroxy-acetylamino)-N-methyl-2,4,6-triiodo-isophthalylamino)-propyl]-N′-(2,3-dihydroxy-propyl)-5-(2-hydroxy-acetylamino)-N′-methyl-2,4,6-triiodo-isophthalamide

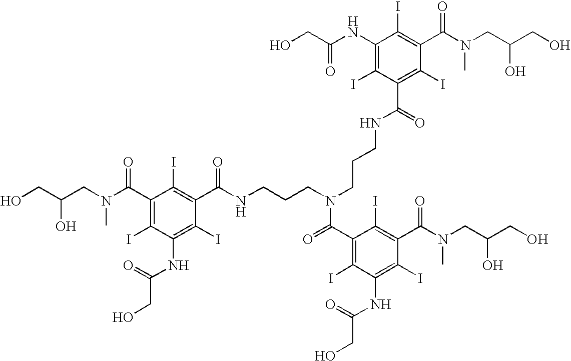

l) N,N-Bis-[3-(N-(2,3-Dihydroxy-propyl)-5-(2,3,4-trihydroxy-butyrylamino)-N-methyl-2,4,6-triiodo-isophthalylamino)-propyl]-N′-(2,3-dihydroxy-propyl)-5-(2,3,4-trihydroxy-butyrylamino)-N′-methyl-2,4,6-triiodo-isophthalamide

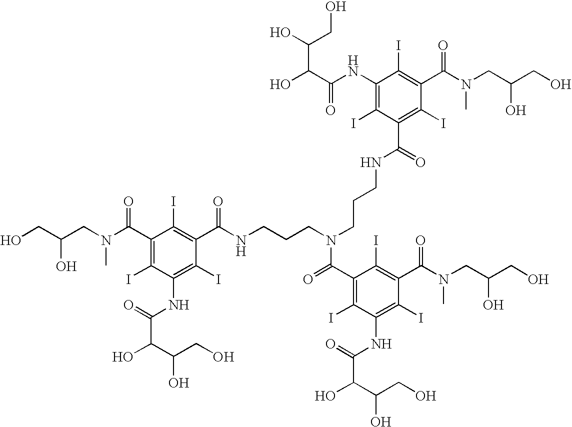

m) N,N-Bis-[3-(N-(2,3-dihydroxy-propyl)-5-(2-hydroxy-acetylamino)-2,4,6-triiodo-isophthalylamino)-3-methyamino-propyl]-N′-(2,3-dihydroxy-propyl)-5-(2-hydroxy-acetylamino)-2,4,6-triiodo-isophthalamide

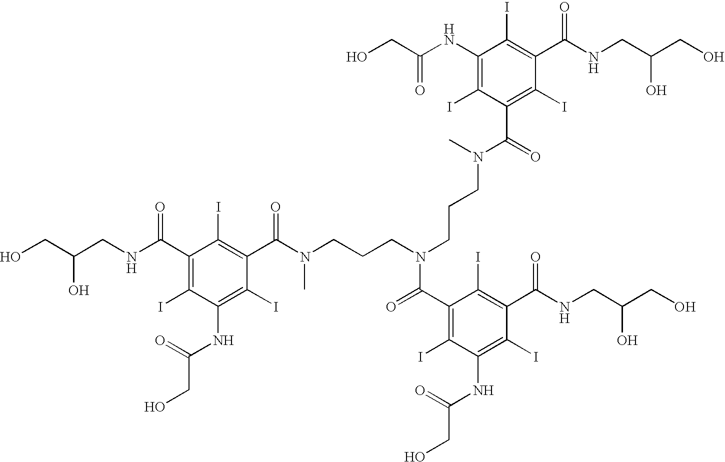

n) N,N-Bis-[3-(N-(2,3-dihydroxy-propyl)-5-(2,3-dihydroxy-propionylamino)-2,4,6-triiodo-isophthalylamino)-3-methylamino-propyl]-N′-(2,3-dihydroxy-propyl)-5-(2,3-Dihydroxy-propionylamino)-2,4,6-triiodo-isophthalamide

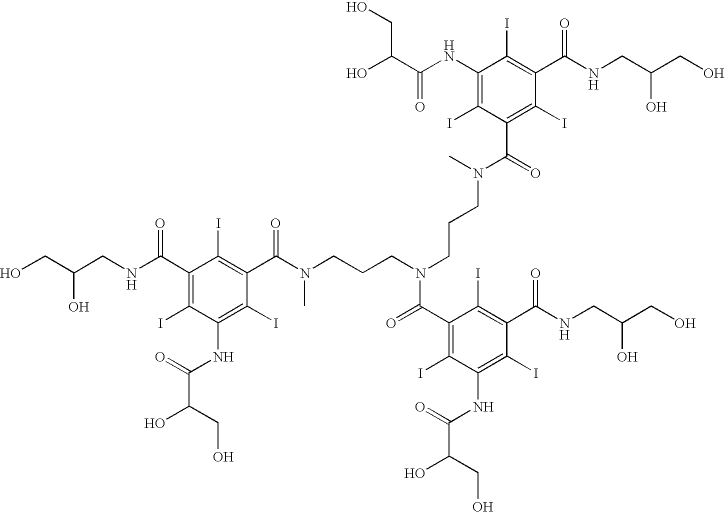

o) N,N-Bis-[3-(N-(2,3-dihydroxy-propyl)-5-(2,3,4-trihydroxy-butyrylamino)-2,4,6-triiodo-isophthalylamino)-3-methylamino-propyl]-N′-(2,3-dihydroxy-propyl)-5-(2,3,4-trihydroxy-butyrylamino)-2,4,6-triiodo-isophthalamide

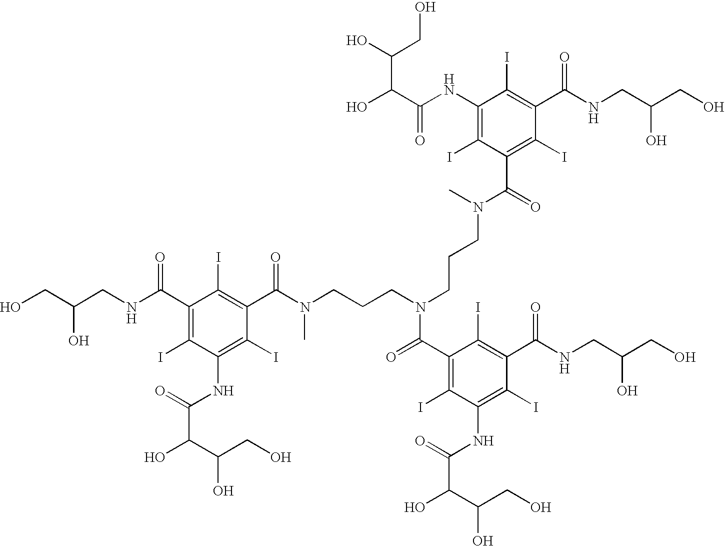

p) N,N-Bis-[3-(N-(2,3-dihydroxy-propyl)-5-(2-hydroxy-acetylamino)-N-methyl-2,4,6-triiodo-isophthalylamino)-3-methylamino-propyl]-N′-(2,3-dihydroxy-propyl)-5-(2-hydroxy-acetylamino)-N′-methyl-2,4,6-triiodo-isophthalamide

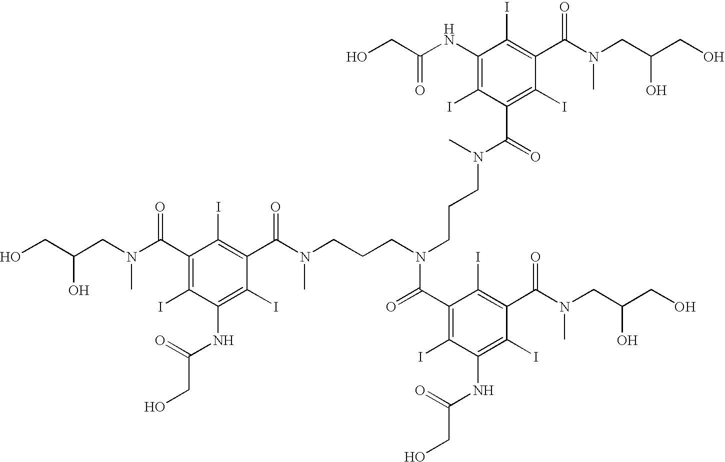

q) N,N-Bis-[3-(N-(2,3-dihydroxy-propyl)-5-(2,3-dihydroxy-propionylamino)-N-methyl-2,4,6-triiodo-isophthalylamino)-3-methylamino-propyl]-N′-(2,3-dihydroxy-propyl)-5-(2,3-dihydroxy-propionylamino)-N′-methyl-2,4,6-triiodo-isophthalamide

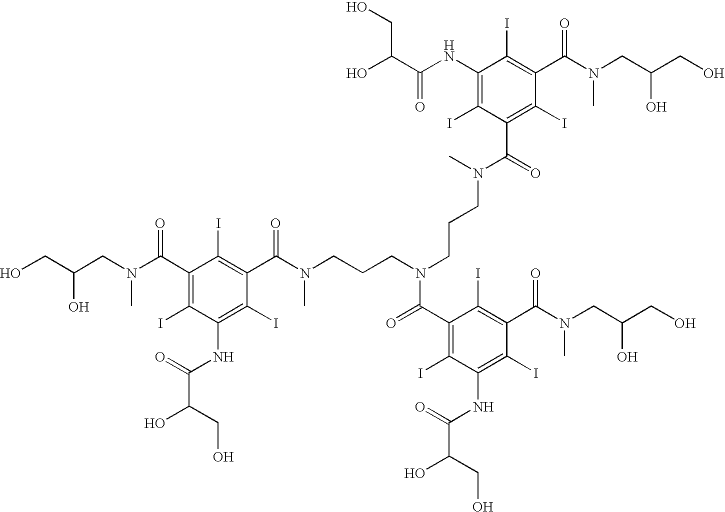

r) N,N-Bis-[3-(N-(2,3-dihydroxy-propyl)-5-(2,3,4-trihydroxy-butyrylamino)-N-methyl-2,4,6-triiodo-isophthalylamino)-3-methylamino-propyl]-N′-(2,3-dihydroxy-propyl)-5-(2,3,4-trihydroxy-butyrylamino)-N′-methyl-2,4,6-triiodo-isophthalamide

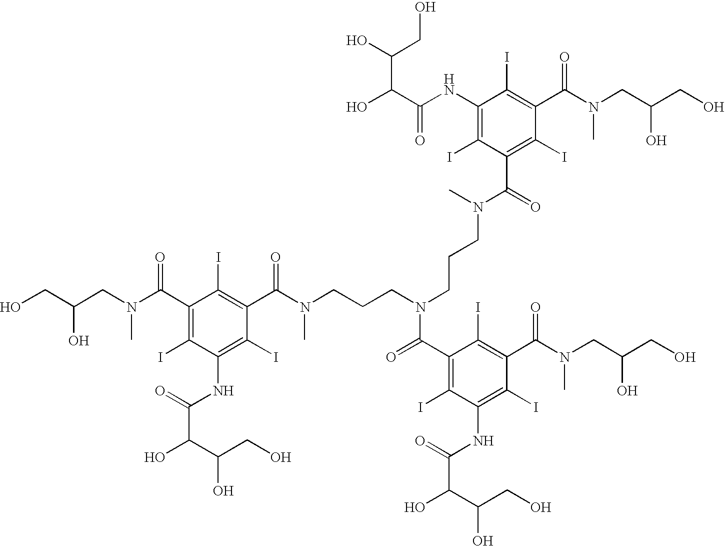

s) N-[2-(N-(2,3-dihydroxy-propyl)-5-(2-hydroxy-acetylamino)-2,4,6-triiodo-isophthalylamino)-ethyl]-N-[3-(N-(2,3-dihydroxy-propyl)-5-(2-hydroxy-acetylamino)-2,4,6-triiodo-isophthalylamino)-propyl]-N′-(2,3-dihydroxy-propyl)-5-(2-hydroxy-acetylamino)-2,4,6-triiodo-isophthalamide

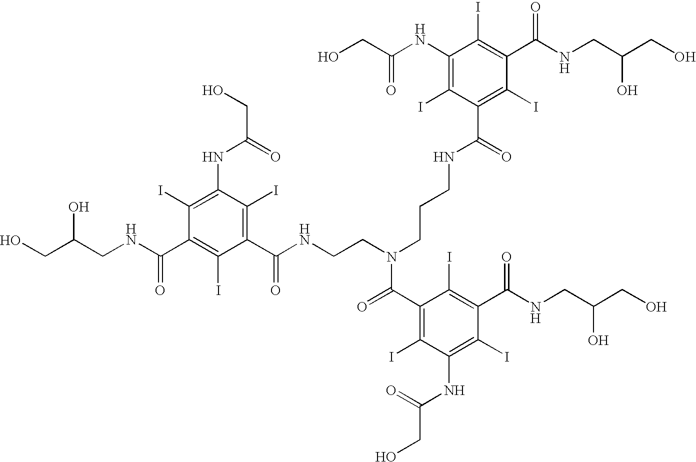

t) N-[2-(N-(2,3-dihydroxy-propyl)-5-(2,3-dihydroxy-propionylamino)-2,4,6-triiodo-isophthalylamino)-ethyl]-N-[3-(N-(2,3-dihydroxy-propyl)-5-(2,3-dihydroxy-propionylamino)-2,4,6-triiodo-isophthalylamino)-propyl]-N′-(2,3-dihydroxy-propyl)-5-(2,3-dihydroxy-propionylamino)-2,4,6-triiodo-isophthalamide

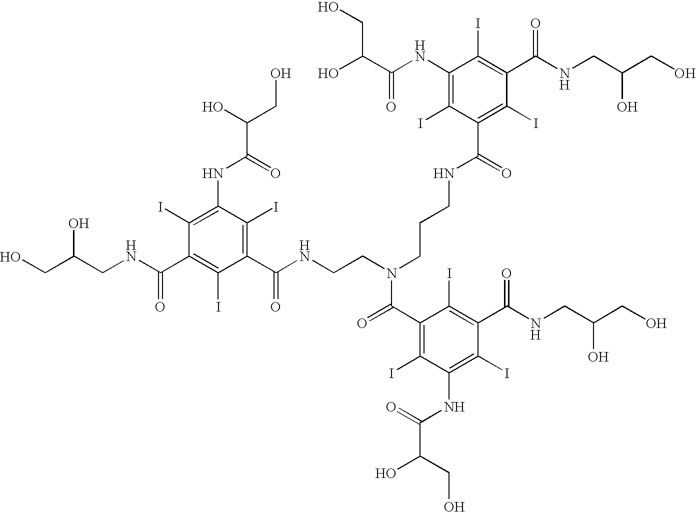

u) N-[2-(N-(2,3-dihydroxy-propyl)-5-(2,3,4-trihydroxy-butyrylamino)-2,4,6-triiodo-isophthalylamino)-ethyl]-N-[3-(N-(2,3-dihydroxy-propyl)-5-(2,3,4-trihydroxy-butyrylamino)-2,4,6-triiodo-isophthalylamino)-propyl]-N′-(2,3-dihydroxy-propyl)-5-(2,3,4-trihydroxy-butyrylamino)-2,4,6-triiodo-isophthalamide

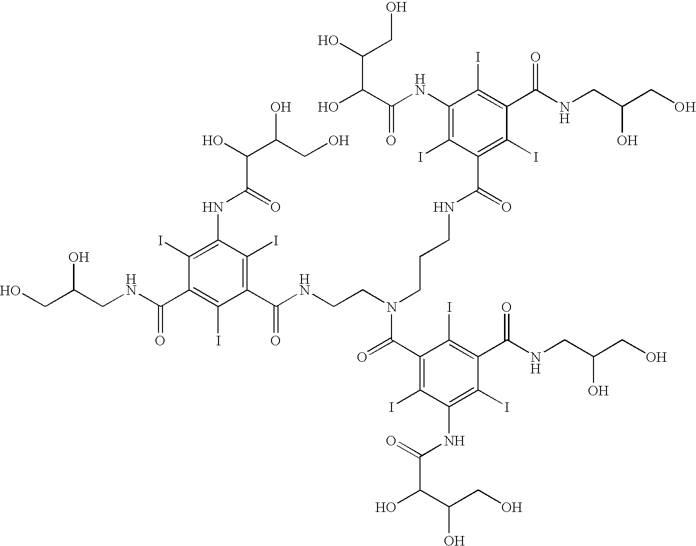

v) N-[2-(N-(2,3-dihydroxy-propyl)-5-(2-hydroxy-acetylamino)-N-methyl-2,4,6-triiodo-isophthalylamino)-ethyl]-N-[3-(N-(2,3-dihydroxy-propyl)-5-(2-hydroxy-acetylamino)-N-methyl-2,4,6-triiodo-isophthalylamino)-propyl]-N′-(2,3-dihydroxy-propyl)-5-(2-hydroxy-acetylamino)-N′-methyl-2,4,6-triiodo-isophthalamide

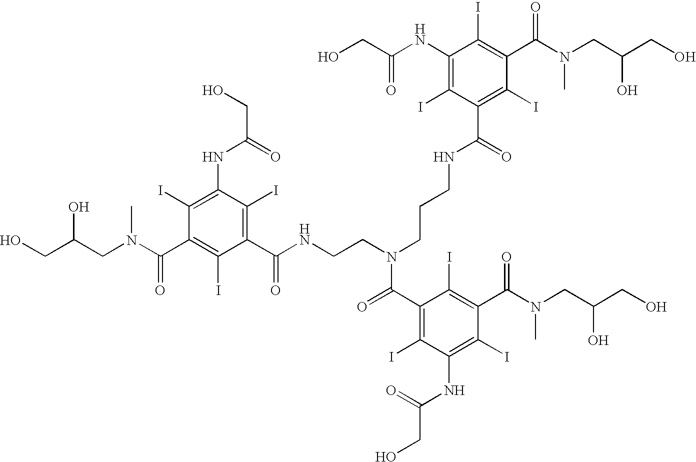

w) N-[2-(N-(2,3-dihydroxy-propyl)-5-(2,3-dihydroxy-propionylamino)-N-methyl-2,4,6-triiodo-isophthalylamino)-ethyl]-N-[3-(N-(2,3-dihydroxy-propyl)-5-(2,3-dihydroxy-propionylamino)-N-methyl-2,4,6-triiodo-isophthalylamino)-propyl]-N′-(2,3-dihydroxypropyl)-5-(2,3-dihydroxy-propionylamino)-N′-methyl-2,4,6-triiodo-isophthalamide

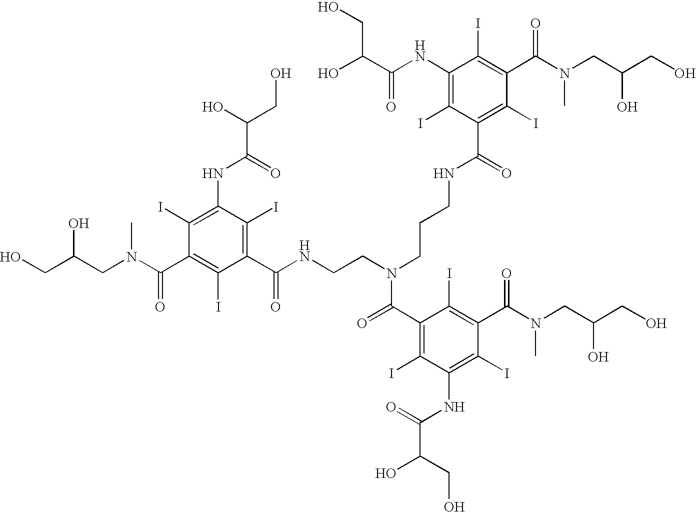

x) N-[2-(N-(2,3-Dihydroxy-propyl)-5-(2,34-trihydroxy-butyrylamino)-N-methyl-2,4,6-triiodo-isophthalylamino)-ethyl]-N-[3-(N-(2,3-Dihydroxy-propyl)-5-(2,3,4-trihydroxy-butyrylamino)-N-methyl-2,4,6-triiodo-isophthalylamino)-propyl]-N′-(2,3-dihydroxy-propyl)-5-(2,3,4-trihydroxy-butyrylamino)-N′-methyl-2,4,6-triiodo-isophthalamide

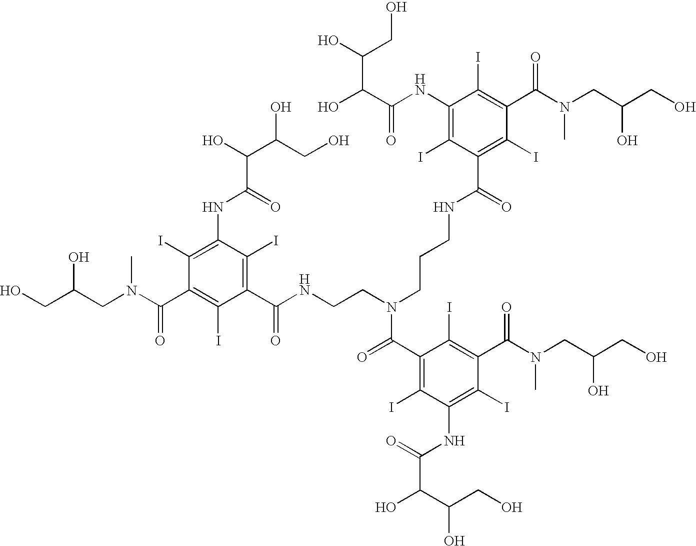

y) N-[2-(N-(2,3-dihydroxy-propyl)-5-(2-hydroxy-acetylamino)-2,4,6-triiodo-isophthalylamino)-2-methyamino-ethyl]-N-[3-(N-(2,3-dihydroxy-propyl)-5-(2-hydroxy-acetylamino)-2,4,6-triiodo-isophthalylamino)-3-methyamino-propyl]-N′-(2,3-dihydroxy-propyl)-5-(2-hydroxy-acetylamino)-2,4,6-triiodo-isophthalamide

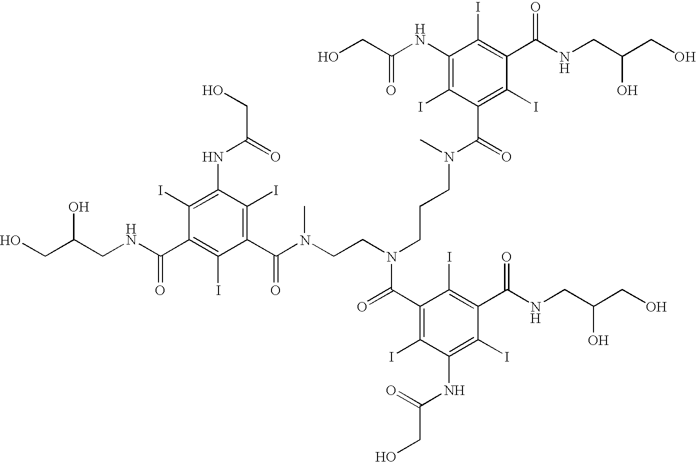

z) N-[2-(N-(2,3-dihydroxy-propyl)-5-(2,3-dihydroxy-propionylamino)-2,4,6-triiodo-isophthalylamino)-2-methylamino-ethyl]-N-[3-(N-(2,3-dihydroxy-propyl)-5-(2,3-dihydroxy-propionylamino)-2,4,6-triiodo-isophthalylamino)-3-methylamino-propyl]-N′-(2,3-dihydroxy-propyl)-5-(2,3-Dihydroxy-propionylamino)-2,4,6-triiodo-isophthalamide

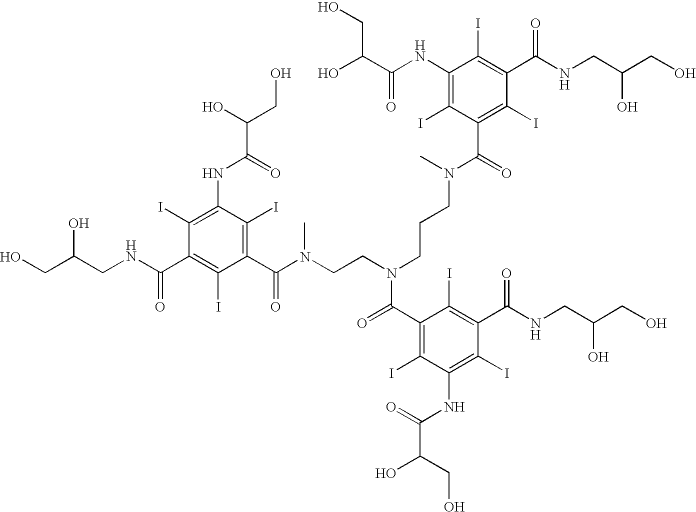

aa) N-[2-(N-(2,3-dihydroxy-propyl)-5-(2,3,4-trihydroxy-butyrylamino)-2,4,6-triiodo-isophthalylamino)-2-methylamino-ethyl]-N-[3-(N-(2,3-dihydroxy-propyl)-5-(2,3,4-trihydroxy-butyrylamino)-2,4,6-triiodo-isophthalylamino)-3-methylamino-propyl]-N′-(2,3-dihydroxy-propyl)-5-(2,3,4-trihydroxy-butyrylamino)-2,4,6-triiodo-isophthalamide

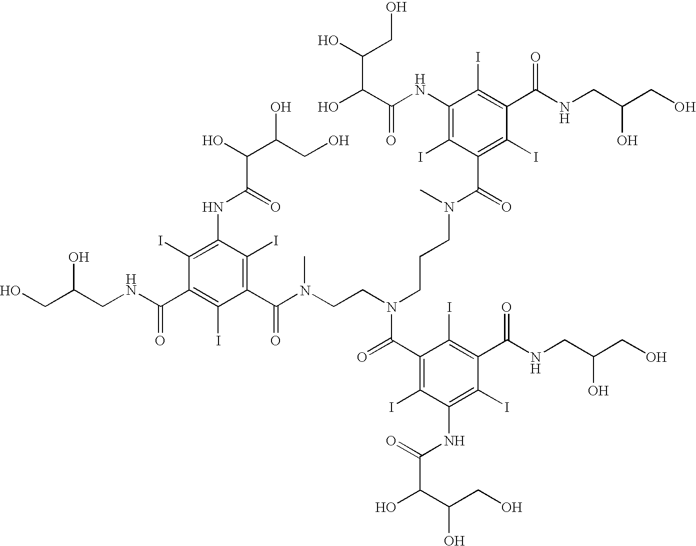

ab) N-[2-(N-(2,3-dihydroxy-propyl)-5-(2-hydroxy-acetylamino)-N-methyl-2,4,6-triiodo-isophthalylamino)-2-methylamino-ethyl]-N-[3-(N-(2,3-dihydroxy-propyl)-5-(2-hydroxy-acetylamino)-N-methyl-2,4,6-triiodo-isophthalylamino)-3-methylamino-propyl]-N′-(2,3-dihydroxy-propyl)-5-(2-hydroxy-acetylamino)-N′-methyl-2,4,6-triiodo-isophthalamide

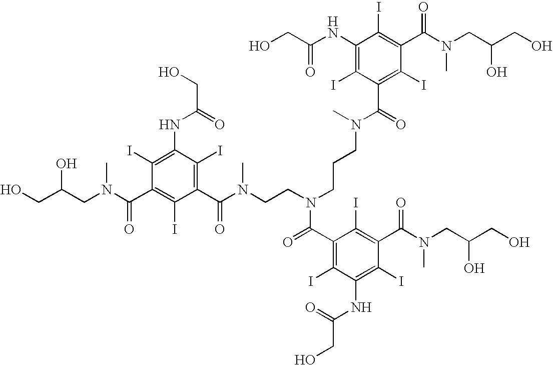

ac) N-[2-(N-(2,3-dihydroxy-propyl)-5-(2,3-dihydroxy-propionylamino)-N-methyl-2,4,6-triiodo-isophthalylamino)-2-methylamino-ethyl]-N-[3-(N-(2,3-dihydroxy-propyl)-5-(2,3-dihydroxy-propionylamino)-N-methyl-2,4,6-triiodo-isophthalylamino)-3-methylamino-propyl]-N′-(2,3-dihydroxy-propyl)-5-(2,3-dihydroxy-propionylamino)-N′-methyl-2,4,6-triiodo-isophthalamide

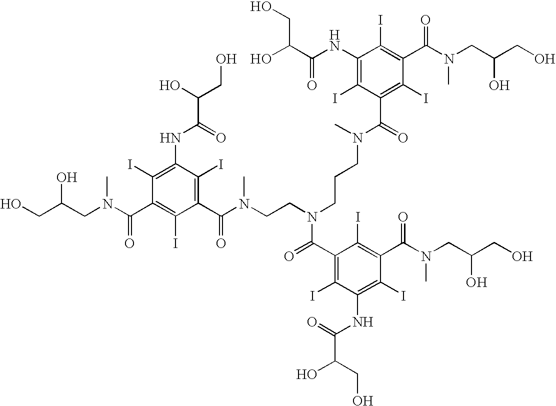

ad) N-[2-(N-(2,3-dihydroxy-propyl)-5-(2,3,4-trihydroxy-butyrylamino)-N-methyl-2,4,6-triiodo-isophthalylamino)-2-methylamino-ethyl]-N-[3-(N-(2,3-dihydroxy-propyl)-5-(2,3,4-trihydroxy-butyrylamino)-N-methyl-2,4,6-triiodo-isophthalylamino)-3-methylamino-propyl]-N′-(2,3-dihydroxy-propyl)-5-(2,3,4-trihydroxy-butyrylamino)-N′-methyl-2,4,6-triiodo-isophthalamide

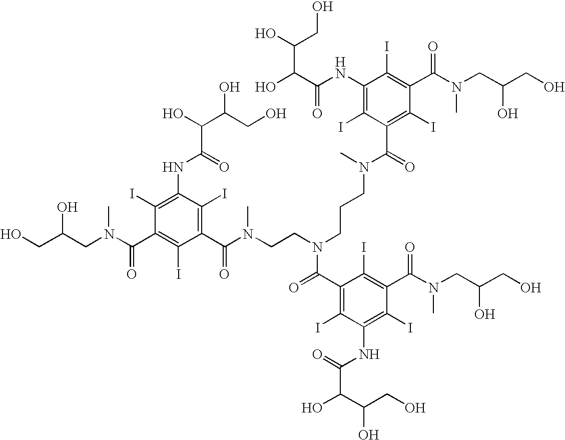

ae) N,N-Bis-[3-(N-(2,3-dihydroxy-propyl)-5-(2-hydroxy-acetylamino)-2,4,6-triiodo-isophthalylamino)-2-hydroxy-propyl]-N′-(2,3-dihydroxy-propyl)-5-(2-hydroxy-acetylamino)-2,4,6-triiodo-isophthalamide

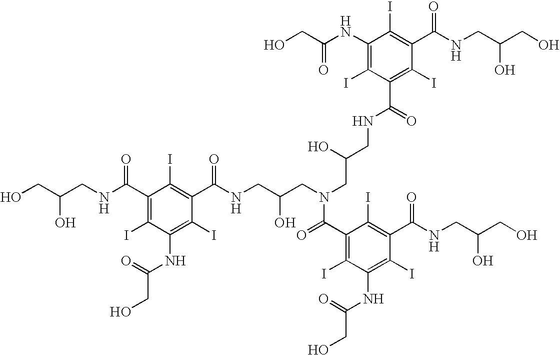

af) N,N-Bis-[3-(N-(2,3-dihydroxy-propyl)-5-(2,3-dihydroxy-propionylamino)-2,4,6-triiodo-isophthalylamino)-2-hydroxy-propyl]-N′-(2,3-dihydroxy-propyl)-5-(2,3-dihydroxy-propionylamino)-2,4,6-triiodo-isophthalamide

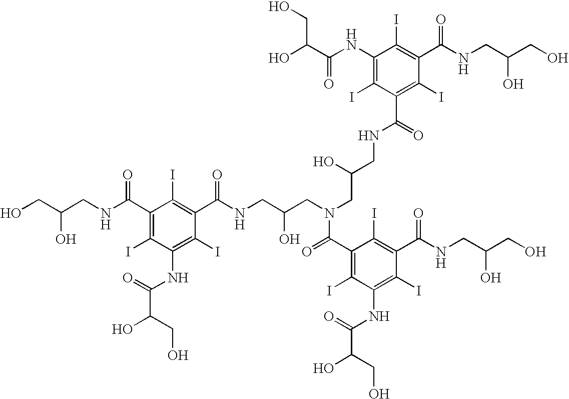

aq) N,N-Bis-[3-(N-(2,3-dihydroxy-propyl)-5-(2,3,4-trihydroxy-butyrylamino)-2,4,6-triiodo-isophthalylamino)-2-hydroxy-propyl]-N′-(2,3-dihydroxy-propyl)-5-(2,3,4-trihydroxy-butyrylamino)-2,4,6-triiodo-isophthalamide

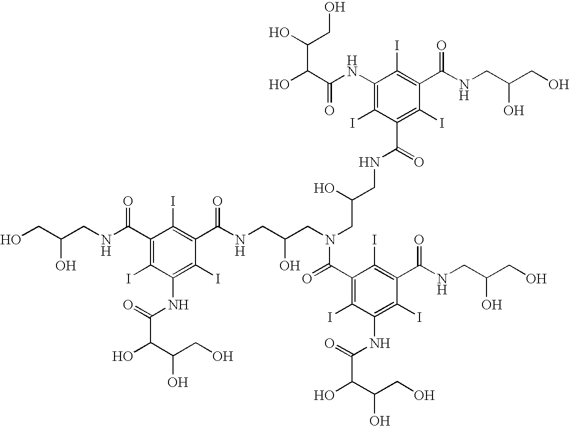

ah) N,N-Bis-[3-(N-(2,3-dihydroxy-propyl)-5-(2-hydroxy-acetylamino)-N-methyl-2,4,6-triiodo-isophthalylamino)-2-hydroxy-propyl]-N′-(2,3-dihydroxy-propyl)-5-(2-hydroxy-acetylamino)-N′-methyl-2,4,6-triiodo-isophthalamide

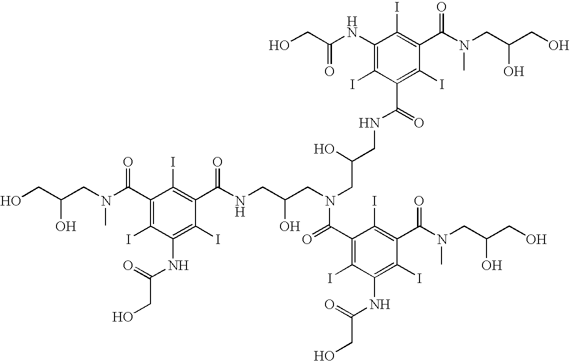

ai) N,N-Bis-[3-(N-(2,3-dihydroxy-propyl)-5-(2,3-dihydroxy-propionylamino)-N-methyl-2,4,6-triiodo-isophthalylamino)-2-hydroxy-propyl]-N′-(2,3-dihydroxy-propyl)-5-(2,3-dihydroxy-propionylamino)-N′-methyl-2,4,6-triiodo-isophthalamide

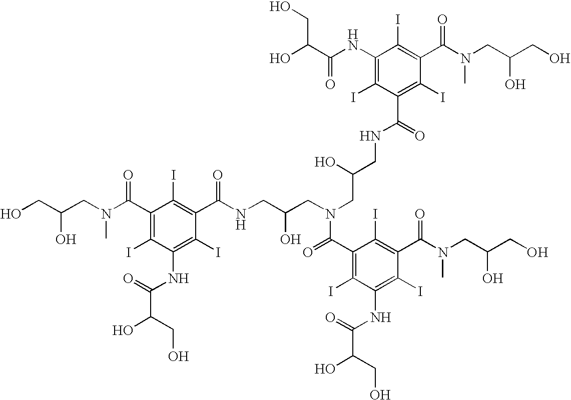

aj) N,N-Bis-[3-(N-(2,3-dihydroxy-propyl)-N-methyl-5-(2,3,4-trihydroxy-butyrylamino)-2,4,6-triiodo-isophthalylamino)-2-hydroxy-propyl]-N′-(2,3-dihydroxy-propyl)-N′-methyl-5-(2,3,4-trihydroxy-butyrylamino)-2,4,6-triiodo-isophthalamide

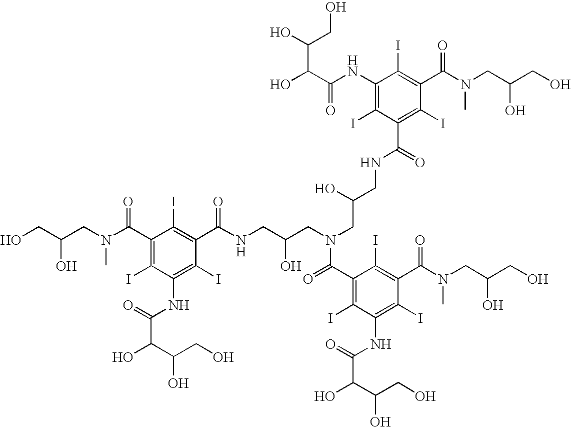

ak) N,N-Bis-[3-(N-(2,3-dihydroxy-propyl)-5-(2-hydroxy-acetylamino)-2,4,6-triiodo-isophthalylamino)-2-hydroxy-3-methylamino-propyl]-N′-(2,3-dihydroxy-propyl)-5-(2-hydroxy-acetylamino)-2,4,6-triiodo-isophthalamide

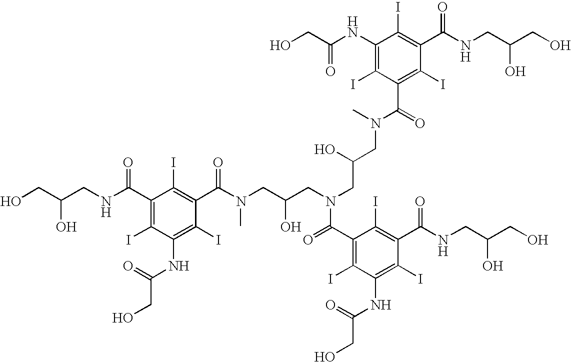

al) N,N-Bis-[3-(N-(2,3-dihydroxy-propyl)-5-(2,3-dihydroxy-propionylamino)-2,4,6-triiodo-isophthalylamino)-2-hydroxy-3-methylamino-propyl]-N′-(2,3-dihydroxy-propyl)-5-(2,3-dihydroxy-propionylamino)-2,4,6-triiodo-isophthalamide

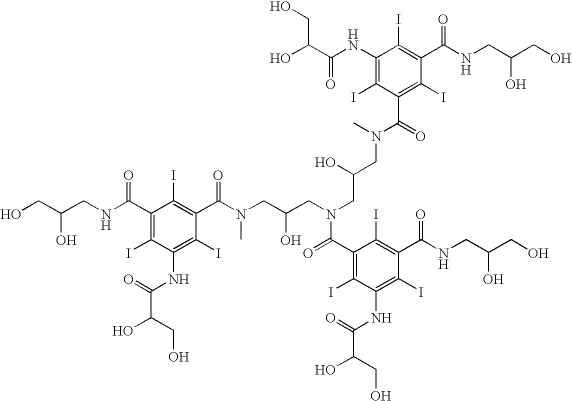

am) N,N-Bis-[3-(N-(2,3-dihydroxy-propyl)-5-(2,3,4-trihydroxy-butyrylamino)-2,4,6-triiodo-isophthalylamino)-2-hydroxy-3-methylamino-propyl]-N′-(2,3-dihydroxy-propyl)-5-(2,3,4-trihydroxy-butyrylamino)-2,4,6-triiodo-isophthalamide

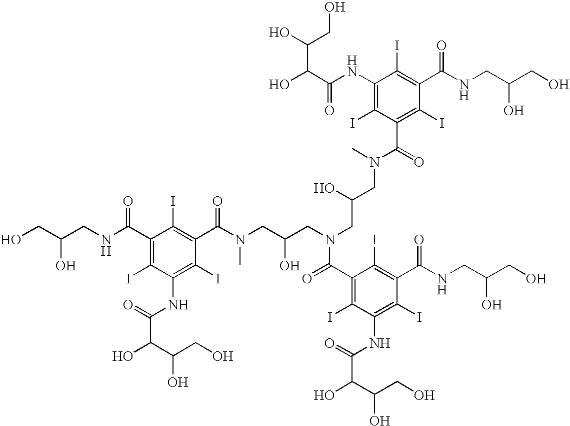

an) N,N-Bis-[3-(N-(2,3-dihydroxy-propyl)-5-(2-hydroxy-acetylamino)-N-methyl-2,4,6-triiodo-isophthalylamino)-2-hydroxy-3-methylamino-propyl]-N′-(2,3-dihydroxy-propyl)-5-(2-hydroxy-acetylamino)-N′-methyl-2,4,6-triiodo-isophthalamide

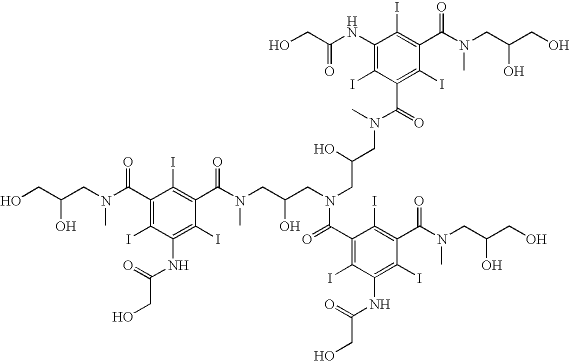

ao) N,N-Bis-[3-(N-(2,3-dihydroxy-propyl)-5-(2,3-dihydroxy-propionylamino)-N-methyl-2,4,6-triiodo-isophthalylamino)-2-hydroxy-3-methylamino-propyl]-N′-(2,3-dihydroxy-propyl)-5-(2,3-Dihydroxy-propionylamino)-N′-methyl-2,4,6-triiodo-isophthalamide

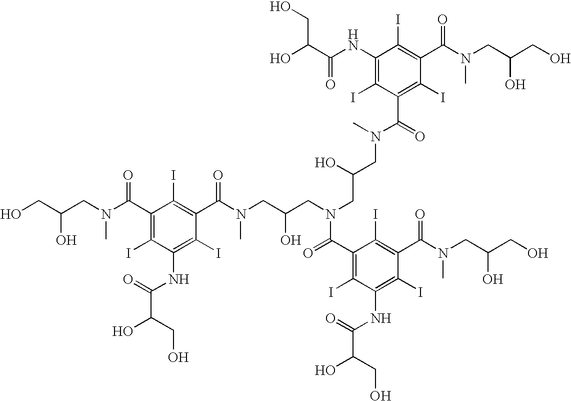

ap) N,N-Bis-[3-(N-(2,3-dihydroxy-propyl)-N-methyl-5-(2,3,4-trihydroxy-butyrylamino)-2,4,6-triiodo-isophthalylamino)-2-hydroxy-3-methylamino-propyl]-N′-(2,3-dihydroxy-propyl)-N′methyl-5-(2,3,4-trihydroxy-butyrylamino)-2,4,6-triiodo-isophthalamide

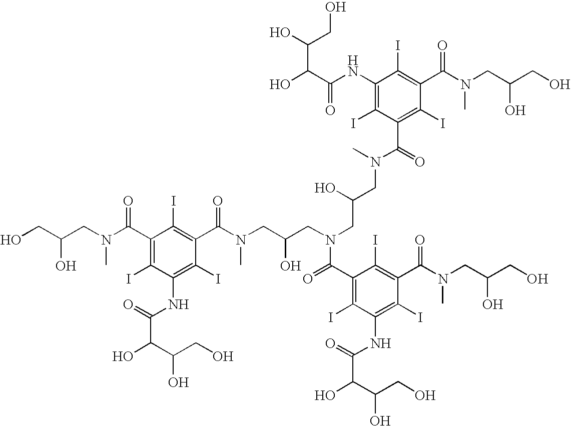

aq) N-[2-(N-(2,3-dihydroxy-propyl)-5-(2-hydroxy-acetylamino)-2,4,6-triiodo-isophthalylamino)-ethyl]-N-[3-(N-(2,3-dihydroxy-propyl)-5-(2-hydroxy-acetylamino)-2,4,6-triiodo-isophthalylamino)-2-hydroxy-propyl]-N′-(2,3-dihydroxy-propyl)-5-(2-hydroxy-acetylamino)-2,4,6-triiodo-isophthalamide

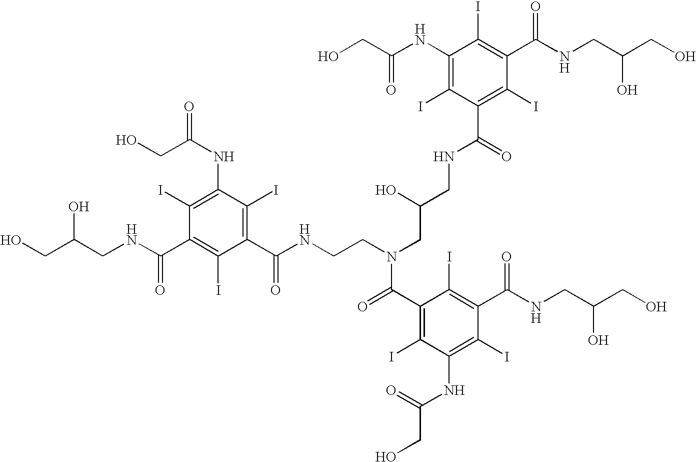

ar) N-[2-(N-(2,3-dihydroxy-propyl)-5-(2,3-dihydroxy-propionylamino)-2,4,6-triiodo-isophthalylamino)-ethyl]-N-[3-(N-(2,3-dihydroxy-propyl)-5-(2,3-dihydroxy-propionylamino)-2,4,6-triiodo-isophthalylamino)-2-hydroxy-propyl]-N′-(2,3-dihydroxy-propyl)-5-(2,3-dihydroxy-propionylamino)-2,4,6-triiodo-isophthalamide

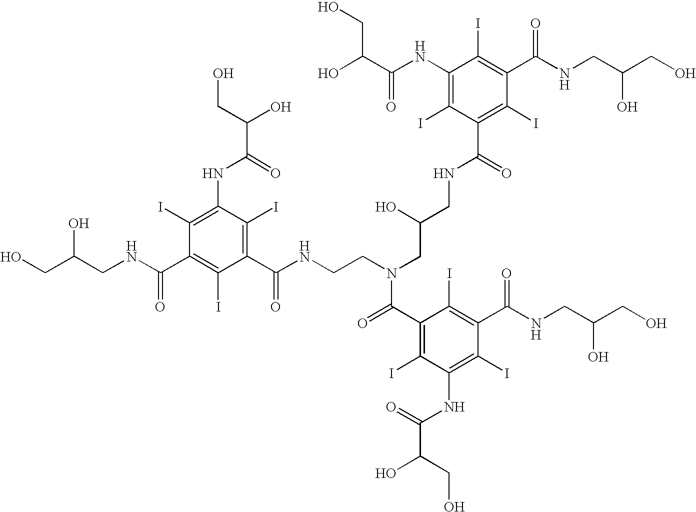

as) N-[2-(N-(2,3-dihydroxy-propyl)-5-(2,3,4-trihydroxy-butyrylamino)-2,4,6-triiodo-isophthalylamino)-ethyl]-N-[3-(N-(2,3-dihydroxy-propyl)-5-(2,3,4-trihydroxy-butyrylamino)-2,4,6-triiodo-isophthalylamino)-2-hydroxy-propyl]-N′-(2,3-dihydroxy-propyl)-5-(2,3,4-trihydroxy-butyrylamino)-2,4,6-triiodo-isophthalamide

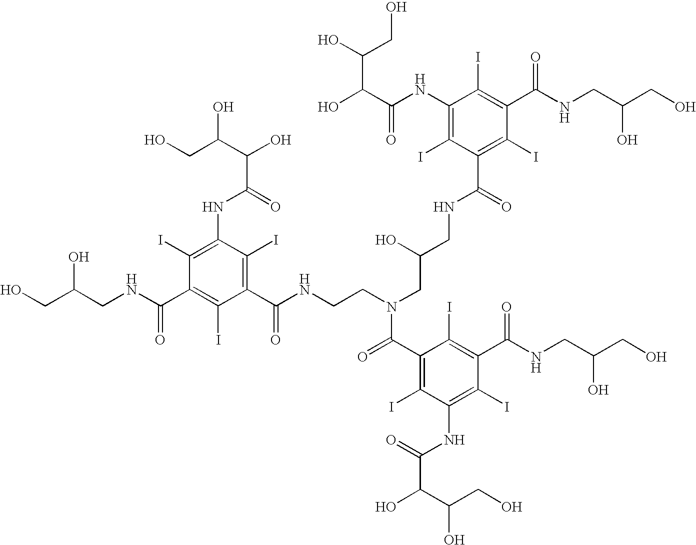

at) N-[2-(N-(2,3-dihydroxy-propyl)-5-(2-hydroxy-acetylamino)-N-methyl-2,4,6-triiodo-isophthalylamino)-ethyl]-N-[3-(N-(2,3-Dihydroxy-propyl)-5-(2-hydroxy-acetylamino)-N-methyl-2,4,6-triiodo-isophthalylamino)-2-hydroxy-propyl]-N′-(2,3-dihydroxy-propyl)-5-(2-hydroxy-acetylamino)-N′-methyl-2,4,6-triiodo-isophthalamide

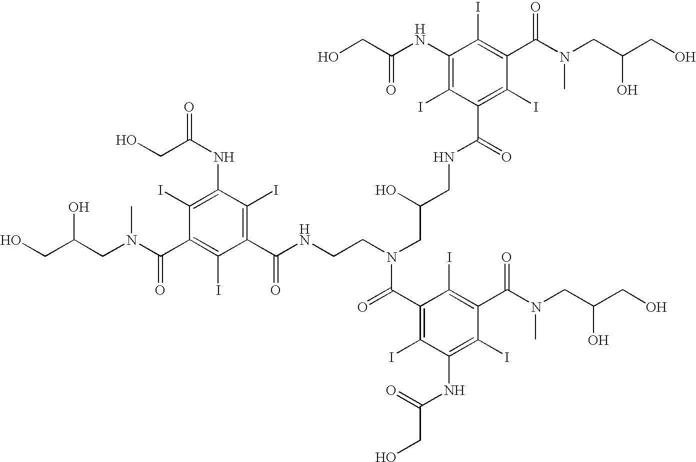

au) N-[2-(N-(2,3-Dihydroxy-propyl)-5-(2,3-dihydroxy-propionylamino)-N-methyl-2,4,6-triiodo-isophthalylamino)-ethyl]-N-[3-(N-(2,3-dihydroxy-propyl)-5-(2,3-dihydroxy-propionylamino)-N-methyl-2,4,6-triiodo-isophthalylamino)-2-hydroxy-propyl]-N′-(2,3-dihydroxy-propyl)-5-(2,3-dihydroxy-propionylamino)-N′-methyl-2,4,6-triiodo-isophthalamide

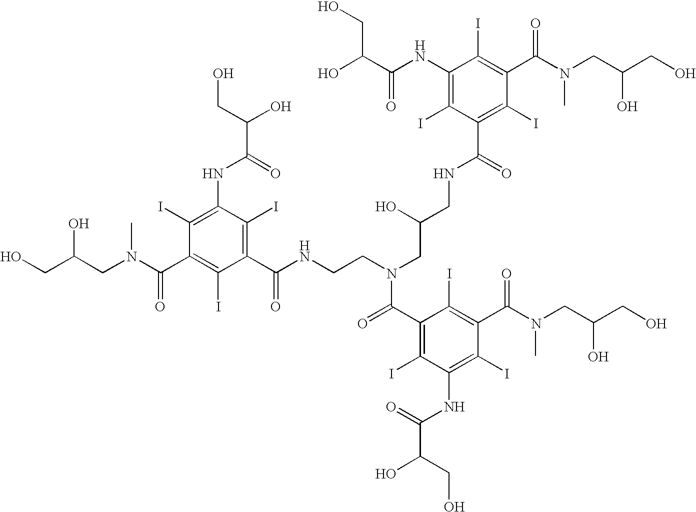

av) N-[2-(N-(2,3-dihydroxy-propyl)-N-methyl-5-(2,3,4-trihydroxy-butyrylamino)-2,4,6-triiodo-isophthalylamino)-ethyl]-N-[3-(N-(2,3-dihydroxy-propyl)-N-methyl-5-(2,3,4-trihydroxy-butyrylamino)-2,4,6-triiodo-isophthalylamino)-2-hydroxy-propyl]-N′-(2,3-dihydroxy-propyl)-N′-methyl-5-(2,3,4-trihydroxy-butyrylamino)-2,4,6-triiodo-isophthalamide

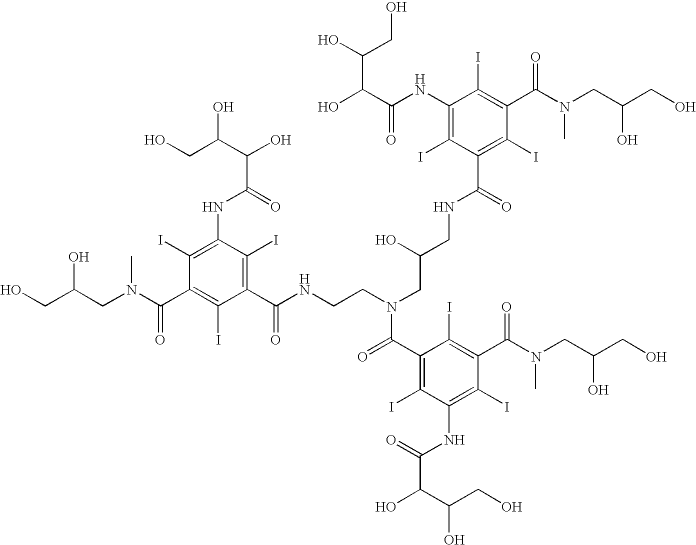

aw) N-[2-(N-(2,3-dihydroxy-propyl)-5-(2-hydroxy-acetylamino)-2,4,6-triiodo-isophthalylamino)-2-methylamino-ethyl]-N-[3-(N-(2,3-dihydroxy-propyl)-5-(2-hydroxy-acetylamino)-2,4,6-triiodo-isophthalylamino)-2-hydroxy-3-methylamino-propyl]-N′-(2,3-dihydroxy-propyl)-5-(2-hydroxy-acetylamino)-2,4,6-triiodo-isophthalamide

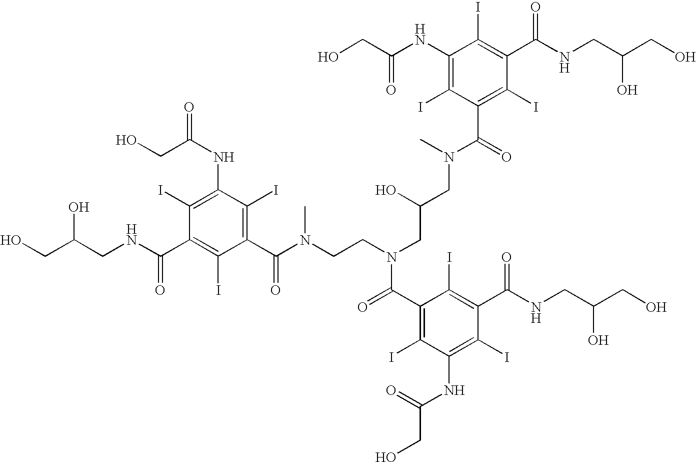

ax) N-[2-(N-(2,3-dihydroxy-propyl)-5-(2,3-dihydroxy-propionylamino)-2,4,6-triiodo-isophthalylamino)-2-methylamino-ethyl]-N-[3-(N-(2,3-dihydroxy-propyl)-5-(2,3-dihydroxy-propionylamino)-2,4,6-triiodo-isophthalylamino)-2-hydroxy-3-methylamino-propyl]-N′-(2,3-dihydroxy-propyl)-5-(2,3-dihydroxy-propionylamino)-2,4,6-triiodo-isophthalamide

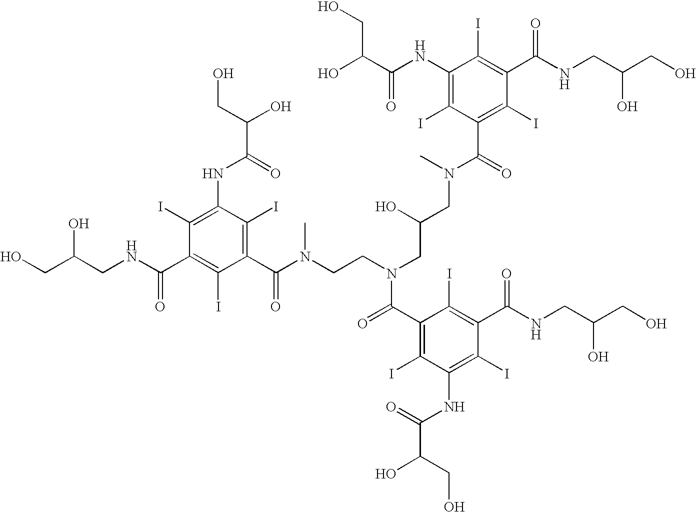

ay) N-[2-(N-(2,3-dihydroxy-propyl)-5-(2,3,4-trihydroxy-butyrylamino)-2,4,6-triiodo-isophthalylamino)-2-methylamino-ethyl]-N-[3-(N-(2,3-dihydroxy-propyl)-5-(2,3,4-trihydroxy-butyrylamino)-2,4,6-triiodo-isophthalylamino)-2-hydroxy-3-methylamino-propyl]-N′-(2,3-dihydroxy-propyl)-5-(2,3,4-trihydroxy-butyrylamino)-2,4,6-triiodo-isophthalamide

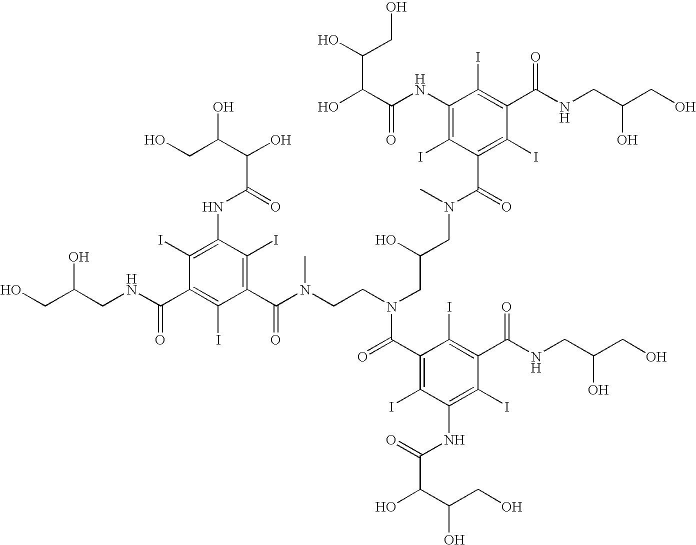

az) N-[2-(N-(2,3-dihydroxy-propyl)-5-(2-hydroxy-acetylamino)-N-methyl-2,4,6-triiodo-isophthalylamino)-2-methylamino-ethyl]-N-[3-(N-(2,3-dihydroxy-propyl)-5-(2-hydroxy-acetylamino)-N-methyl-2,4,6-triiodo-isophthalylamino)-2-hydroxy-3-methylamino-propyl]-N′-(2,3-dihydroxy-propyl)-5-(2-hydroxy-acetylamino)-N′-methyl-2,4,6-triiodo-isophthalamide

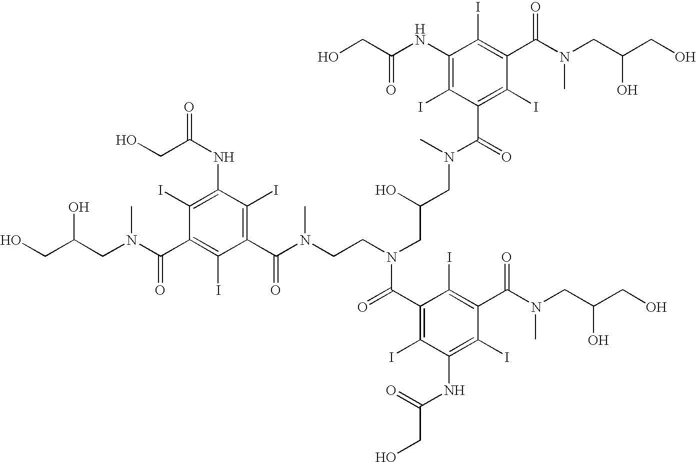

ba) N-[2-(N-(2,3-dihydroxy-propyl)-5-(2,3-dihydroxy-propionylamino)-N-methyl-2,4,6-triiodo-isophthalylamino)-2-methylamino-ethyl]-N-[3-(N-(2,3-dihydroxy-propyl)-5-(2,3-dihydroxy-propionylamino)-N-methyl-2,4,6-triiodo-isophthalylamino)-2-hydroxy-3-methylamino-propyl]-N′-(2,3-dihydroxy-propyl)-5-(2,3-dihydroxy-propionylamino)-N′-methyl-2,4,6-triiodo-isophthalamide

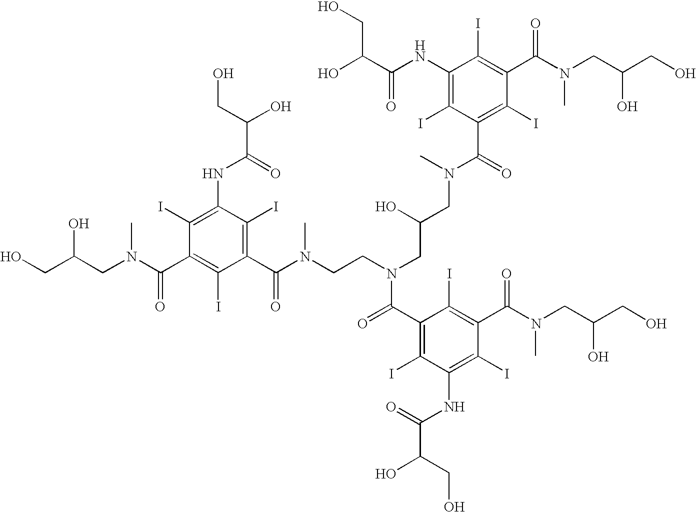

bb) N-[2-(N-(2,3-dihydroxy-propyl)-N-methyl-5-(2,3,4-trihydroxy-butyrylamino)-2,4,6-triiodo-isophthalylamino)-2-methylamino-ethyl]-N-[3-(N-(2,3-dihydroxy-propyl)-N-methyl-5-(2,3,4-trihydroxy-butyrylamino)-2,4,6-triiodo-isophthalylamino)-2-hydroxy-3-methylamino-propyl]-N′-(2,3-dihydroxy-propyl)-N′-methyl-5-(2,3,4-trihydroxy-butyrylamino)-N′-methyl-2,4,6-triiodo-isophthalamide

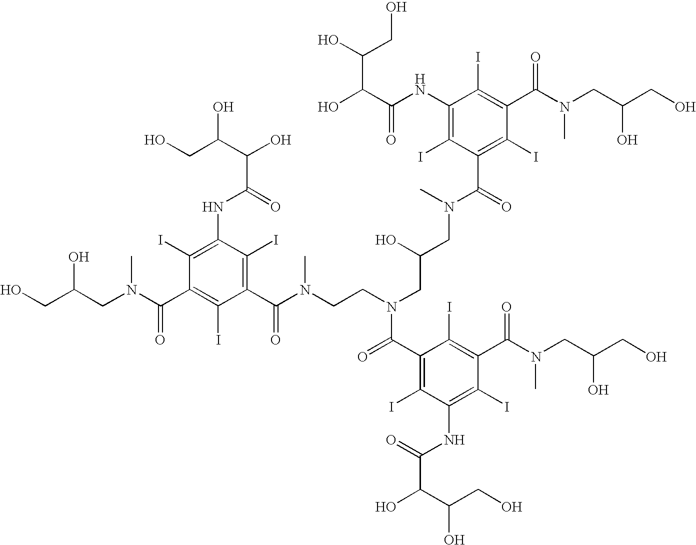

bc) N-[2-(N-(2,3-dihydroxy-propyl)-5-(2-hydroxy-acetylamino)-2,4,6-triiodo-isophthalylamino)-propyl]-N-[3-(N-(2,3-dihydroxy-propyl)-5-(2-hydroxy-acetylamino)-2,4,6-triiodo-isophthalylamino)-2-hydroxy-propyl]-N′-(2,3-dihydroxy-propyl)-5-(2-hydroxy-acetylamino)-2,4,6-triiodo-isophthalamide

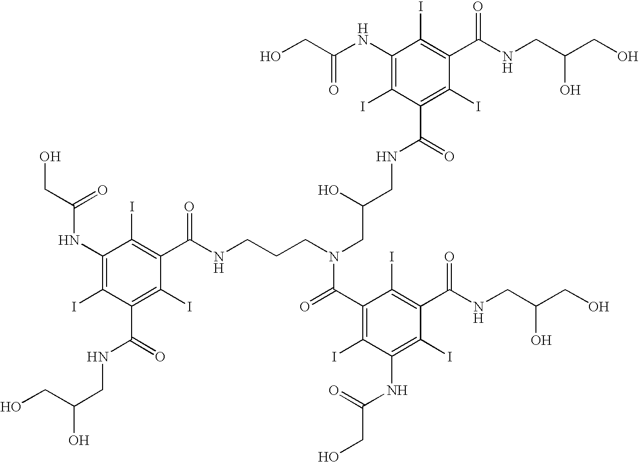

bd) N-[2-(N-(2,3-dihydroxy-propyl)-5-(2,3-dihydroxy-propionylamino)-2,4,6-triiodo-isophthalylamino)-propyl]-N-[3-(N-(2,3-dihydroxy-propyl)-5-(2,3-dihydroxy-propionylamino)-2,4,6-triiodo-isophthalylamino)-2-hydroxy-propyl]-N′-(2,3-dihydroxy-propyl)-5-(2,3-dihydroxy-propionylamino)-2,4,6-triiodo-isophthalamide

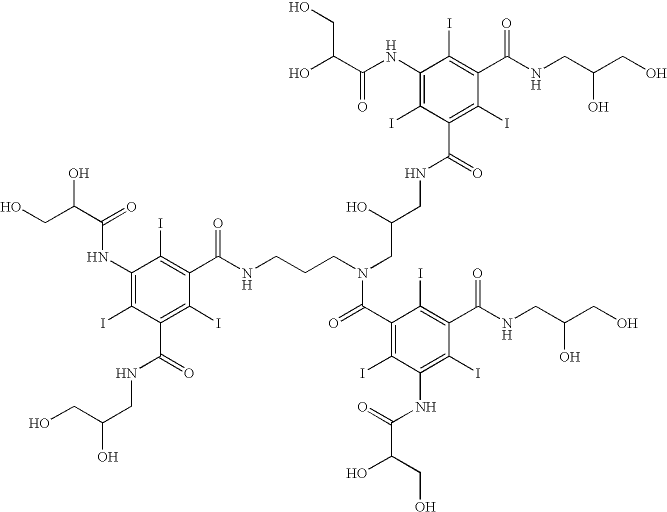

be) N-[2-(N-(2,3-dihydroxy-propyl)-5-(2,3,4-trihydroxy-butyrylamino)-2,4,6-triiodo-isophthalylamino)-propyl]-N-[3-(N-(2,3-dihydroxy-propyl)-5-(2,3,4-trihydroxy-butyrylamino)-2,4,6-triiodo-isophthalylamino)-2-hydroxy-propyl]-N′-(2,3-dihydroxy-propyl)-5-(2,3,4-trihydroxy-butyrylamino)-2,4,6-triiodo-isophthalamide

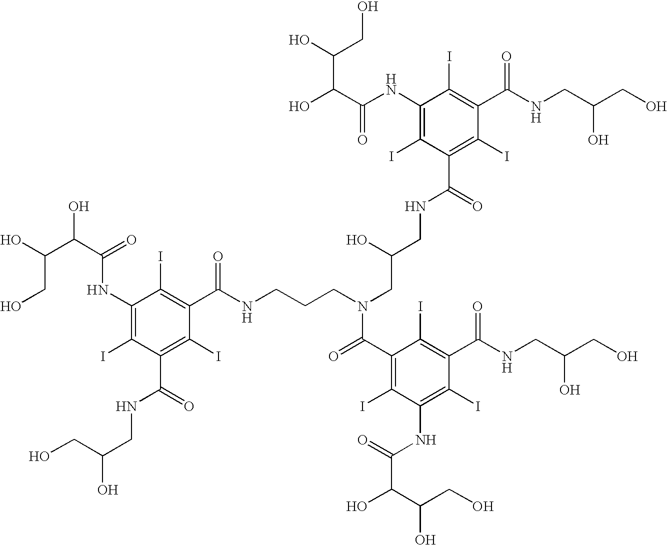

bf) N-[2-(N-(2,3-dihydroxy-propyl)-5-(2-hydroxy-acetylamino)-N-methyl-2,4,6-triiodo-isophthalylamino)-propyl]-N-[3-(N-(2,3-Dihydroxy-propyl)-5-(2-hydroxy-acetylamino)-N-methyl-2,4,6-triiodo-isophthalylamino)-2-hydroxy-propyl]-N′-(2,3-dihydroxy-propyl)-5-(2-hydroxy-acetylamino)-N′-methyl-2,4,6-triiodo-isophthalamide

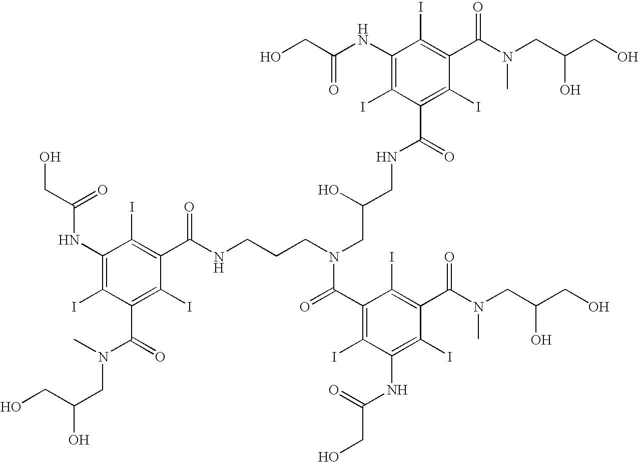

bg) N-[2-(N-(2,3-Dihydroxy-propyl)-5-(2,3-dihydroxy-propionylamino)-N-methyl-2,4,6-triiodo-isophthalylamino)-propyl]-N-[3-(N-(2,3-dihydroxy-propyl)-5-(2,3-dihydroxy-propionylamino)-N-methyl-2,4,6-triiodo-isophthalylamino)-2-hydroxy-propyl]-N-(2,3-dihydroxy-propyl)-5-(2,3-dihydroxy-propionylamino)-N′-methyl-2,4,6-triiodo-isophthalamide

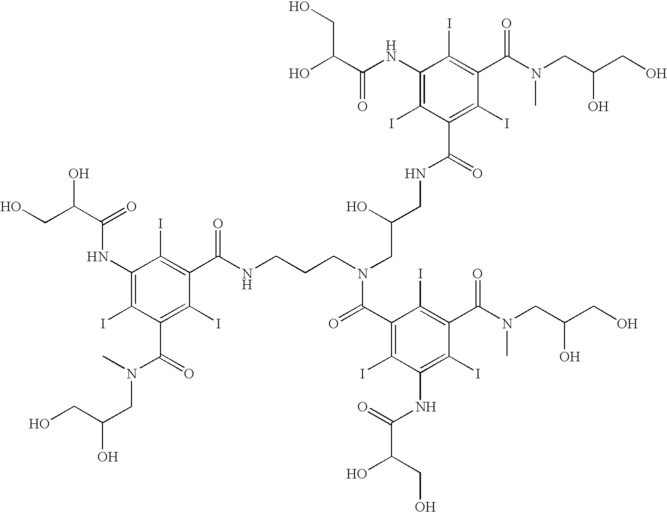

bh) N-[2-(N-(2,3-dihydroxy-propyl)-N-methyl-5-(2,3,4-trihydroxy-butyrylamino)-2,4,6-triiodo-isophthalylamino)-propyl]-N-[3-(N-(2,3-dihydroxy-propyl)-N-methyl-5-(2,3,4-trihydroxy-butyrylamino)-2,4,6-triiodo-isophthalylamino)-2-hydroxy-propyl]-N′-(2,3-dihydroxy-propyl)-N′-methyl-5-(2,3,4-trihydroxy-butyrylamino)-2,4,6-triiodo-isophthalamide

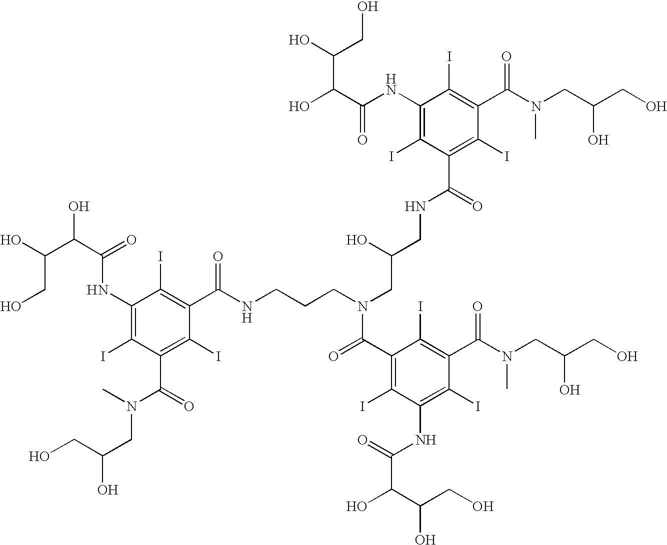

bi) N-[2-(N-(2,3-dihydroxy-propyl)-5-(2-hydroxy-acetylamino)-2,4,6-triiodo-isophthalylamino)-2-methylamino-propyl]-N-[3-(N-(2,3-dihydroxy-propyl)-5-(2-hydroxy-acetylamino)-2,4,6-triiodo-isophthalylamino)-2-hydroxy-3-methylamino-propyl]-N′-(2,3-dihydroxy-propyl)-5-(2-hydroxy-acetylamino)-2,4,6-triiodo-isophthalamide

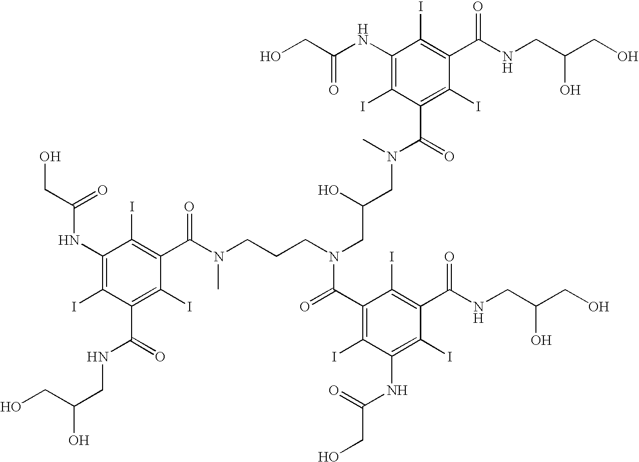

bj) N-[2-(N-(2,3-dihydroxy-propyl)-5-(2,3-dihydroxy-propionylamino)-2,4,6-triiodo-isophthalylamino)-2-methylamino-propyl]-N-[3-(N-(2,3-dihydroxy-propyl)-5-(2,3-dihydroxy-propionylamino)-2,4,6-triiodo-isophthalylamino)-2-hydroxy-3-methylamino-propyl]-N′-(2,3-dihydroxy-propyl)-5-(2,3-dihydroxy-propionylamino)-2,4,6-triiodo-isophthalamide

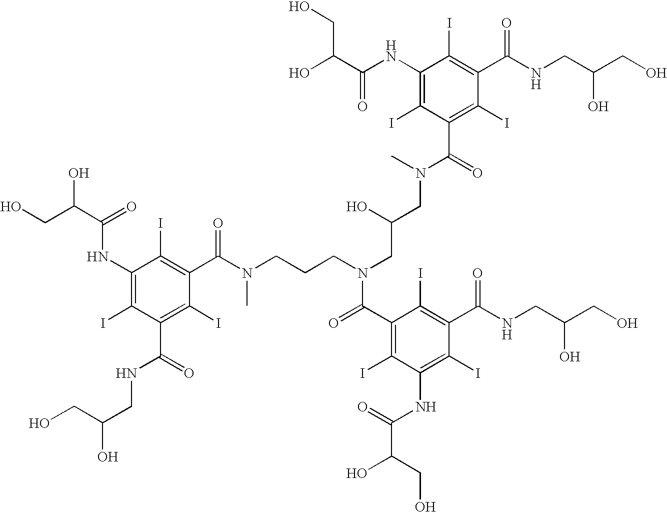

bk) N-[2-(N-(2,3-dihydroxy-propyl)-5-(2,3,4-trihydroxy-butyrylamino)-2,4,6-triiodo-isophthalylamino)-2-methylamino-propyl]-N-[3-(N-(2,3-dihydroxy-propyl)-5-(2,3,4-trihydroxy-butyrylamino)-2,4,6-triiodo-isophthalylamino)-2-hydroxy-3-methylamino-propyl]-N′-(2,3-dihydroxy-propyl)-5-(2,3,4-trihydroxy-butyrylamino)-2,4,6-triiodo-isophthalamide

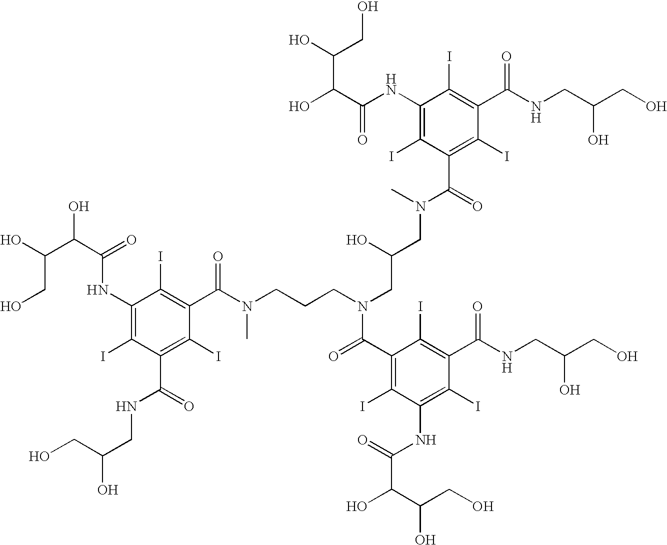

bl) N-[2-(N-(2,3-dihydroxy-propyl)-5-(2-hydroxy-acetylamino)-N-methyl-2,4,6-triiodo-isophthalylamino)-2-methylamino-propyl]-N-[3-(N-(2,3-dihydroxy-propyl)-5-(2-hydroxy-acetylamino)-N-methyl-2,4,6-triiodo-isophthalylamino)-2-hydroxy-3-methylamino-propyl]-N′-(2,3-dihydroxy-propyl)-5-(2-hydroxy-acetylamino)-N′-methyl-2,4,6-triiodo-isophthalamide

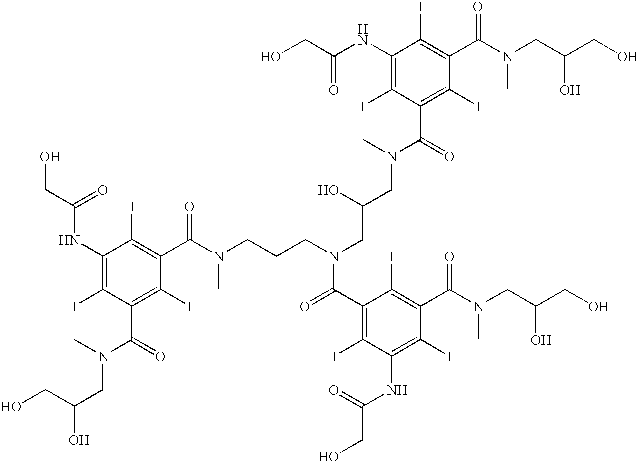

bm) N-[2-(N-(2,3-dihydroxy-propyl)-5-(2,3-dihydroxy-propionylamino)-N-methyl-2,4,6-triiodo-isophthalylamino)-2-methylamino-propyl]-N-[3-(N-(2,3-dihydroxy-propyl)-5-(2,3-dihydroxy-propionylamino)-N-methyl-2,4,6-triiodo-isophthalylamino)-2-hydroxy-3-methylamino-propyl]-N′-(2,3-dihydroxy-propyl)-5-(2,3-dihydroxy-propionylamino)-N′-methyl-2,4,6-triiodo-isophthalamide

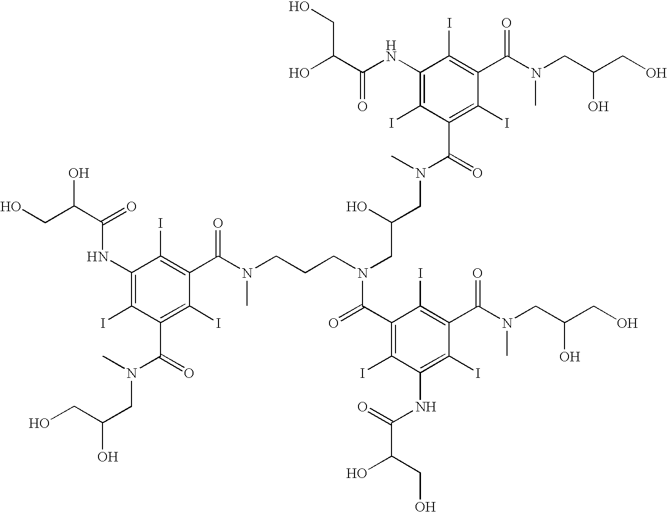

bn) N-[2-(N-(2,3-dihydroxy-propyl)-N-methyl-5-(2,3,4-trihydroxy-butyrylamino)-2,4,6-triiodo-isophthalylamino)-2-methylamino-propyl]-N-[3-(N-(2,3-dihydroxy-propyl)-N-methyl-5-(2,3,4-trihydroxy-butyrylamino)-2,4,6-triiodo-isophthalylamino)-2-hydroxy-3-methylamino-propyl]-N′-(2,3-dihydroxy-propyl)-N′-methyl-5-(2,3,4-trihydroxy-butyrylamino)-N′-methyl-2,4,6-triiodo-isophthalamide

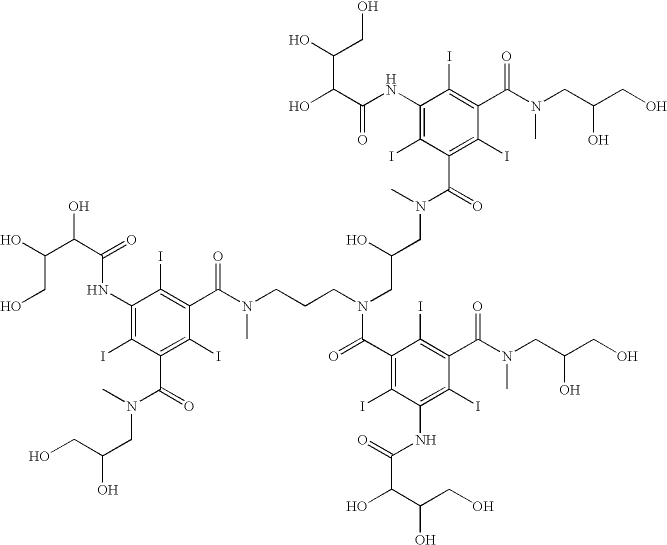

### Example 8

### N,N-Bis-[2-(N,N-bis(2,3-dihydroxy-propyl)-5-(2,3-dihydroxy-propionylamino)-2,4,6-triiodo-isophthalylamino)-ethyl]-N′,N′-bis(2,3-dihydroxy-propyl)-5-(2,3-dihydroxy-propionylamino)-2,4,6-triiodo-isophthalamide

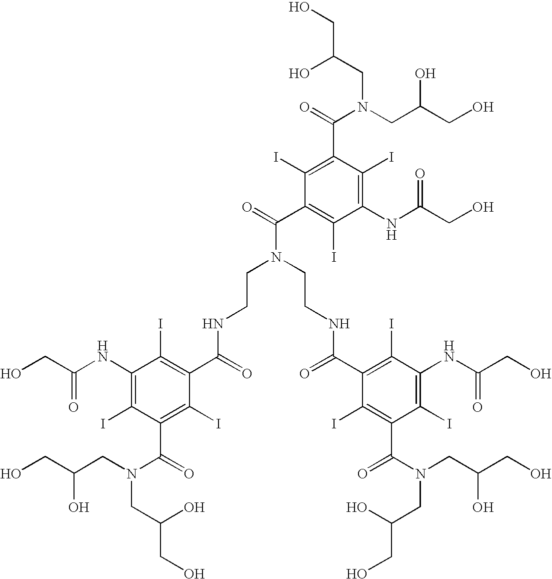

This compound was produced following the procedure of example 1. The crude product was purified by preparative reverse phase HPLC (C18 Luna column: 10 micron, 250×50 mm), eluting with water (A) and acetonitrile (B) (5% B→30% B in 10 min) with a flow of 120 mL/min, to afford 625 mg (39%) of the compound. The structure was confirmed by NMR. LC-MS: calcd for C₅₂H₆₄I₉N₉O₂₄ 2340.5; found, 1171.6 (M/2+H)⁺.

### Example 9

### N,N-Bis-[2-(N-(2,3-dihydroxy-propyl)-5-(2,3-dihydroxy-propionylamino)-2,4,6-triiodo-isophthalylamino)-ethyl]-N′-(2,3-dihydroxy-propyl)-5-(2,3-dihydroxy-propionylamino)-2,4,6-triiodo-isophthalamide

### a) Synthesis of acetic acid 2-acetoxy-1-{3-chlorocarbonyl-5-[(2,2-dimethyl-[1,3]dioxolan-4-ylmethyl)carbamoyl]-2,4,6-triiodophenylcarbamoyl}ethyl ester

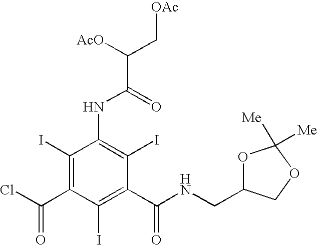

In a round bottom flask, 15 g of Acetic acid 2-acetoxy-1-(3,5-bis-chlorocarbonyl-2,4,6-triiodo-phenylcarbamoyl)-ethyl ester (15, g, 19.5 mmol) was dissolved in dry DMAc (75 mL). To this solution was added 5.0 mL of 2,2-dimethyl-1,3-dioxolane-4-methanamine (5.0 mL, 39 mmol). The reaction mixture was stirred overnight at room temperature. The mixture was concentrated to dryness to give the crude product, which was purified by column chromatography packed with silica gel (330 g) eluting with petroleum ether (A) and ethyl acetate (B) (20% B→100% B) for 10 CV at a flow of 150 mL/min, to afford 12 g (71%) of acetic acid 2-acetoxy-1-{3-chlorocarbonyl-5-[(2,2-dimethyl-[1,3]dioxolan-4-ylmethyl)carbamoyl]-2,4,6-triiodophenylcarbamoyl}ethyl ester. The structure was confirmed by ¹H NMR (300 MHz, DMSO-d6): δ 1.33 (s, 3H), 1.43 (s, 3H), 2.08 (s, 3H), 2.28 (s, 3H), 3.42-3.40 (m, 1H), 3.78-3.65 (m, 2H), 4.35-4.30 (m, 1H), 4.50-4.45 (m, 1H), 4.64-4.60 (m, 1H), 5.63 (br s, 1H), 6.39 (br s, 1H). LC-MS: calcd for C₂₁H₂₂ClI₃N₂O₉ 861.8; found, 862.7 (M+H)⁺.

### b) Synthesis of acetic acid 2-acetoxy-1-{3-chlorocarbonyl-5-[(2,2-dimethyl-[1,3]dioxolan-4-ylmethyl)carbamoyl]-2,4,6-triiodophenylcarbamoyl}ethyl ester

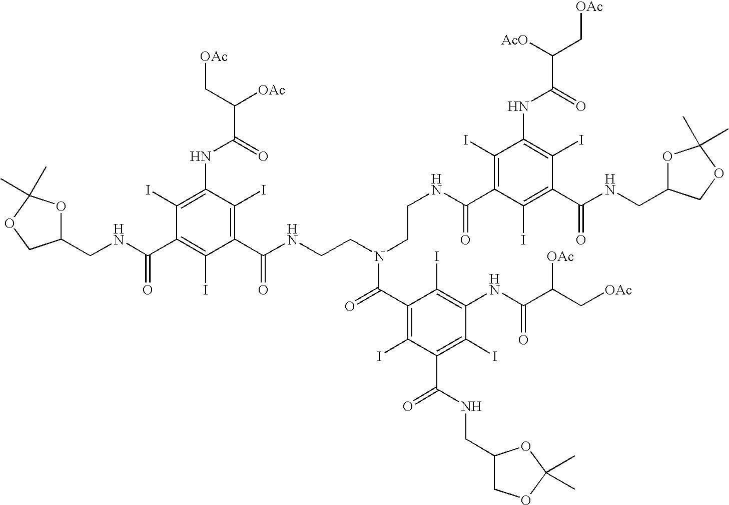

In a round bottom flask, acetic acid 2-acetoxy-1-{3-chlorocarbonyl-5-[(2,2-dimethyl-[1,3]dioxolan-4-ylmethyl)carbamoyl]-2,4,6-triiodophenylcarbamoyl}ethyl ester (4 g, 4.64 mmol) was dissolved in DMAc (15 mL). To this solution was added diethylenetriamine (153 mg, 1.5 mmol, 160 μL) as triamine, followed by triethylamine (472 mg, 4.6 mmol, 650 μL). The reaction mixture was heated at 60° C. overnight. The mixture was concentrated to dryness to give the crude product, which was purified by column chromatography packed with silica gel (120 g column) eluting with ethyl acetate (A) and methanol (B) (2.5% B→30% B) for 40 CV at a flow of 85 mL/min, to afford 277 mg (8.5%) of the trimer. LC-MS: calcd for C₆₇H₇₆I₉N₉O₂₇ 2580.6; found, 1291.4 (M/2+H)⁺.

### c) Synthesis of N,N-Bis-[2-(N-(2,3-dihydroxy-propyl)-5-(2,3-dihydroxy-propionylamino)-2,4,6-triiodo-isophthalylamino)-ethyl]-N′-(2,3-dihydroxy-propyl)-5-(2,3-dihydroxy-propionylamino)-2,4,6-triiodo-isophthalamide

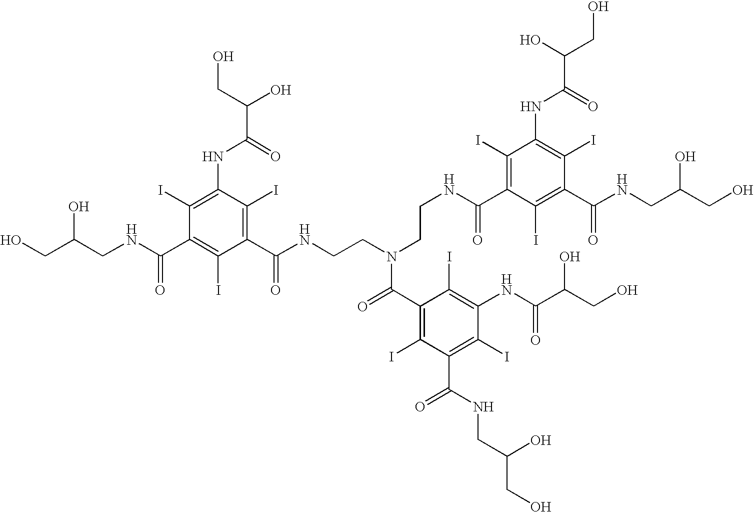

In a round bottom flask, acetic acid 2-acetoxy-1-{3-chlorocarbonyl-5-[(2,2-dimethyl-[1,3]dioxolan-4-ylmethyl)carbamoyl]-2,4,6-triiodophenylcarbamoyl}ethyl ester was dissolved in methanol (20 mL). To this solution was added a solution of HCl_{(aq)} (10 mL) (1M). The reaction mixture was refluxed for 1 h. The mixture was concentrated to dryness to give the crude product, which was purified by preparative reverse phase HPLC (C18 Luna column: 5 micron, 250×21.2 mm), eluting with water (A) and acetonitrile (B) (5% B→25% B in 20 min) with a flow of 21 mL/min, to afford 277 mg (8.5%) of N,N-Bis-[2-(N-(2,3-dihydroxy-propyl)-5-(2,3-dihydroxy-propionylamino)-2,4,6-triiodo-isophthalylamino)-ethyl]-N′-(2,3-dihydroxy-propyl)-5-(2,3-dihydroxy-propionylamino)-2,4,6-triiodo-isophthalamide. The structure was confirmed by NMR. LC-MS: calcd for C₄₆H₅₂I₉N₉O₂₁ 2208.5; found, 1105.3 (M/2+H)⁺.

What is claimed is:

1. Compounds of formula (I) 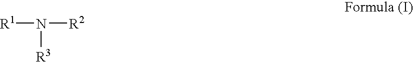
and salts or optical active isomers thereof
wherein R¹ and R² independently are the same or different and denote a moiety —X—NR⁴—CO—R;
each R³ denote a moiety —CO—R;
each R⁴ are the same or different and denote a hydrogen atom, hydroxyl group or a C₁-C₄ alkyl group where the alkyl group may be substituted by hydroxyl and amino groups and interrupted by an oxygen atom;
each X are the same or different and denote a straight or branched C₁-C₄ alkylene group where the alkylene group may be substituted by hydroxyl and amino groups and interrupted by an oxygen atom; and
each R independently are the same or different and denote a triiodinated phenyl group further substituted by two groups R⁵ wherein each R⁵ are the same or different and denote a hydrogen atom or a non-ionic hydrophilic moiety, provided that at least one R⁵ group in the compound of formula (I) is a hydrophilic moiety.

2. Compound as claimed in claim 1 wherein R¹ and R² independently of each other denote the moiety —(CH₂)ₙ—NR⁴—CO—R wherein n denote an integer of 1 to 4.

3. Compound as claimed in claim 2 wherein n denote an integer of 2 and 3.

4. Compound as claimed in claim 1 wherein R⁴ denotes a hydrogen atom or an unsubstituted C₁-C₄ alkyl group.

5. Compound as claimed in claim 4 wherein R⁴ denotes a hydrogen atom or a methyl group.

6. Compound as claimed in claim 1 wherein the R¹ and R² groups are the same.

7. Compound as claimed in claim 1 wherein the R¹ and R³ groups are the same.

8. Compound as claimed in claim 1 wherein each R are the same or different and denote a 2,4,6 triiodinated phenyl group, further substituted by two groups R⁵.

9. Compound as claimed in claim 1 wherein each R⁵ are the same or different and denote a non-ionic hydrophilic moiety comprising esters, amides and amine moieties, optionally further substituted by a straight chain or branched chain C₁₋₁₀ alkyl groups, optionally with one or more CH₂ or CH moieties replaced by oxygen or nitrogen atoms and optionally substituted by one or more groups selected from oxo, hydroxyl, amino or carboxyl derivative, and oxo substituted sulphur and phosphorus atoms.

10. Compound as claimed in claim 9 wherein each R⁵ are the same or different and denote a non-ionic hydrophilic moiety comprising esters, amides and amine moieties, optionally further substituted by a straight chain or branched chain C₁₋₅ alkyl groups, optionally with one or more CH₂ or CH moieties replaced by oxygen or nitrogen atoms and optionally substituted by one or more groups selected from oxo, hydroxyl, amino or carboxyl derivative, and oxo substituted sulphur and phosphorus atoms.

11. Compound as claimed in claim 10 wherein each R⁵ are the same or different and denote a non-ionic hydrophilic moiety comprising esters, amides and amine moieties, further substituted by a straight chain or branched chain C₁₋₅ alkyl groups substituted by 1 to 3 hydroxy groups.

12. Compound as claimed in claim 11 wherein each R⁵ are the same or different and are polyhydroxy C₁₋₅ alkyl, hydroxyalkoxyalkyl with 1 to 5 carbon atoms, and hydroxypolyalkoxyalkyl with 1 to 5 carbon atoms, attached to the iodinated phenyl group via an amide or a carbamoyl linkage.

13. Compound as claimed in claim 1 wherein each R⁵ are the same or different and is selected from the following group:
—CONH—CH₂—CH₂—OH,
—CONN—CH₂—CHOH—CH₂—OH,
—CON(CH₃)CH₂—CHOH—CH₂OH,
—CONH—CH—(CH₂—OH)₂,
—CON—(CH₂—CH₂—OH)₂,
—CON—(CH₂—CHOH—CH₂—OH)₂,
—CONH₂,
—CONHCH₃,
—CONH—CH₂—CH₂—O—CH₃,
—CONH—O—CH₃,
—CONH—CH₂—CHOH—CH₂—O—CH₃,
—CONH—CH₂—CHOCH₃—CH₂—OH,
—CON(CH₂—CHOH—CH₂—OH)(CH₂—CH₂—OH),
—CONH—C(CH₂—OH)₂CH₃,
—CONH—C(CH₂—OH)₃,
—CONH—CH(CH₂—OH)(CHOH—CH₂—OH),
—N(COCH₃)H,
—N(COCH₃)C₁₋₃alkyl,
—N(COCH₃)—mono, bis or tris-hydroxy C₁₋₄ alkyl,
—N(COCH₂OH)—hydrogen, mono, bis or tris-hydroxy C₁₋₄ alkyl,
—N(CO—CHOH—CH₂OH)—hydrogen, mono, bis or trihydroxylated C₁₋₄ alkyl,
—N(CO—CHOH—CHOH—CH₂OH)—hydrogen, mono, bis or trihydroxylated C₁₋₄ alkyl,
—N(CO—CH—(CH₂OH)₂)—hydrogen, mono, bis or trihydroxylated C₁₋₄ alkyl, and
—N(COCH₂OH)₂.

14. Compound as claimed in claim 13 wherein each R⁵ are the same or different and are selected from groups —CONH—CH₂—CHOH—CH₂—OH, —CONH—CH—(CH₂—OH)₂, —CON—(CH₂—CH₂—OH)₂, —CON—(CH₂—CHOH—CH₂—OH)₂, —CON(CH₃)—CH₂—CHOH—CH₂—OH, —NH—COCH₂OH, —NH—CO—CHOH—CH₂OH, —NH—CO—CHOH—CHOH—CH₂OH, and —N(COCH₂OH)—mono, bis or tris-hydroxy C₁₋₄ alkyl.

15. Compound as claimed in claim 1 wherein in all R groups are the same and each R⁵ are different and are selected from groups —CONH—CH₂—CHOH—CH₂—OH, —CON(CH₃)—CH₂—CHOH—CH₂—OH, —CON—(CH₂—CHOH—CH₂—OH)₂, —NH—COCH₂OH, —NH—CO—CHOH—CH₂OH and —NH—CO—CHOH—CHOH—CH₂OH.

16. Compound as claimed in claim 1 of formula (II) 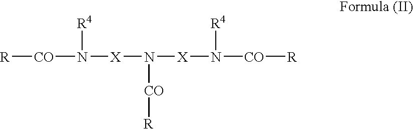
wherein each group R, R⁴ and X are as defined in claim 1.

17. Compound as claimed in claim 1 selected from:
N,N-Bis-[2-(N-(2,3-dihydroxy-propyl)-5-(2-hydroxy-acetylamino)-2,4,6-triiodo-isophthalylamino)-ethyl]-N′-(2,3-dihydroxy-propyl)-5-(2-hydroxy-acetylamino)-2,4,6-triiodo-isophthalamide;
N,N-Bis-[2-(N-(2,3-dihydroxy-propyl)-5-(2-hydroxy-acetylamino)-N-methyl-2,4,6-triiodo-isophthalylamino)-ethyl]-N′-(2,3-dihydroxy-propyl)-5-(2-hydroxy-acetylamino)-N′-methyl-2,4,6-triiodo-isophthalamide;
N,N-Bis-[2-(N-(2,3-dihydroxy-propyl)-5-(2,3-dihydroxy-propionylamino)-N-methyl-2,4,6-triiodo-isophthalylamino)-ethyl]-N′-(2,3-dihydroxy-propyl)-5-(2,3-dihydroxy-propionylamino)-N′-methyl-2,4,6-triiodo-isophthalamide;
N,N-Bis-[2-(N-(2,3-dihydroxy-propyl)-N-methyl-5-(2,3,4-trihydroxy-butyrylamino)-2,4,6-triiodo-isophthalylamino)-ethyl]-N′-(2,3-dihydroxy-propyl)-N′-methyl-5-(2,3,4-trihydroxy-butyrylamino)-2,4,6-triiodo-isophthalamide;
N,N-Bis-[2-(N-(2,3-dihydroxy-propyl)-N-methyl-5-(2,3,4-trihydroxy-butyrylamino)-2,4,6-triiodo-isophthalylamino)-2-methylamino-ethyl]-N′-(2,3-dihydroxy-propyl)-N′-methyl-5-(2,3,4-trihydroxy-butyrylamino)-2,4,6-triiodo-isophthalamide;
N,N-Bis-[3-(N-(2,3-dihydroxy-propyl)-5-(2,3-dihydroxy-propionylamino)-N-methyl-2,4,6-triiodo-isophthalylamino)-propyl]-N′-(2,3-dihydroxy-propyl)-5-(2,3-dihydroxy-propionylamino)-N′-methyl-2,4,6-triiodo-isophthalamide;
N-[2-(N-(2,3-dihydroxy-propyl)-5-(2,3-dihydroxy-propionylamino)-N-methyl-2,4,6-triiodo-isophthalylamino)-propyl]-N-[3-(N-(2,3-dihydroxy-propyl)-5-(2,3-dihydroxy-propionylamino)-N-methyl-2,4,6-triiodo-isophthalylamino)-butyl]-N′-(2,3-dihydroxy-propyl)-5-(2,3-dihydroxy-propionylamino)-N′-methyl-2,4,6-triiodo-isophthalamide;
N,N-Bis-[2-(N,N-bis(2,3-dihydroxy-propyl)-5-(2,3-dihydroxy-propionylamino)-2,4,6-triiodo-isophthalylamino)-ethyl]-N′,N′-bis(2,3-dihydroxy-propyl)-5-(2,3-dihydroxy-propionylamino)-2,4,6-triiodo-isophthalamide; and
N,N-Bis-[2-(N-(2,3-dihydroxy-propyl)-5-(2,3-dihydroxy-propionylamino)-2,4,6-triiodo-isophthalylamino)-ethyl]-N′-(2,3-dihydroxy-propyl)-5-(2,3-dihydroxy-propionylamino)-2,4,6-triiodo-isophthalamide.

18. A diagnostic agent comprising a compound of formula (I) as defined in claim 1.

19. A diagnostic composition comprising a compound of formula (I) as defined in claim 1 together with pharmaceutically acceptable carriers or excipients.

20. An X-ray diagnostic composition comprising a compound of formula (I) as defined in claim 1 together with pharmaceutically acceptable carriers or excipients.

21. (canceled)

22. (canceled)

23. A method of diagnosis comprising administration of a compound of formula (I) as defined in claim 1 to the human or animal body, examining the body with a diagnostic device and compiling data from the examination.

24. A method of diagnosis comprising examining a body preadministered with a compound of formula (I) as defined in claim 1 with a diagnostic device and compiling data from the examination.

25. A method of imaging, specifically X-ray imaging, comprising administration of a compound of formula (I) as defined in claim 1 to the human or animal body, examining the body with a diagnostic device, compiling data from the examination, and optionally analysing the data.
